(19) United States
(12) Patent Application Publication
(10) Pub. No.: US 2016/0244769 A1
(43) Pub. Date: Aug. 25, 2016

(54) MICROBIAL HEXOSE FORMATION

(71) Applicant: UNIVERSITY OF GEORGIA RESEARCH FOUNDATION, INC., Athens, GA (US)

(72) Inventors: Tian Xia, Athens, GA (US); Mark A. Eiteman, Athens, GA (US)

(73) Assignee: UNIVERSITY OF GEORGIA RESEARCH FOUNDATION, INC., Athens, GA (US)

(21) Appl. No.: 15/053,533

(22) Filed: Feb. 25, 2016

Related U.S. Application Data
(60) Provisional application No. 62/120,725, filed on Feb. 25, 2015.

(51) Int. Cl. C12N 15/70; C12P 19/02

(52) U.S. Cl. CPC C12N 15/70; C12P 19/02

(57) ABSTRACT

The present invention provides cells that are metabolically engineered for formation and accumulation of a hexose or other glucose-6P or fructose-6P metabolite, as well methods for making said cells and methods for forming and isolating the hexose or other metabolite.

This application claims the benefit of U.S. Provisional Application Ser. No. 62/120,725, filed Feb. 25, 2015, which is incorporated herein by reference in its entirety.

### GOVERNMENT FUNDING

This invention was made with government support under CBET-0929893, awarded by the National Science Foundation. The government has certain rights in the invention.

### SEQUENCE LISTING

This application contains a Sequence Listing electronically submitted to the United States Patent and Trademark Office via EFS-Web as an ASCII text file entitled “2016-02-25-SequenceListing_ST25.txt” having a size of 1 KB and created on 22 February 2016. Due to the electronic filing of the Sequence Listing, the electronically submitted Sequence Listing serves as both the paper copy required by 37 CFR §1.821(c) and the CRF required by §1.821(e). The information contained in the Sequence Listing is incorporated by reference herein.

### BACKGROUND

Glucose is the primary currency of energy for much of life. Plant, bacterial and mammalian cells have evolved highly efficient biochemical pathways to oxidize glucose, not only to generate energy but also to synthesize precursor molecules used as the building blocks of cellular materials. Many cells have also devised mechanisms to store glucose in different forms: plants generate starch and cellulose, while many eukaryotic cells accumulate glycogen. In addition to precursor molecules and storage products, structurally diverse compounds are derived from glucose and other monosaccharides; for example, glycosylation of small molecules and proteins provides unique cellular functionalities.

### SUMMARY OF THE INVENTION

The present invention provides cells that are metabolically engineered for formation and accumulation of a metabolite of glucose-6P or fructose-6P, such as a hexose, as well methods for making said cells and methods for forming and isolating said metabolites from said cells or cell culture. The term “glucose-6P or fructose-6P metabolite” includes but is not limited to any compound containing a carbohydrate moiety that is enzymatically formed within the metabolically engineered cell from the compounds glucose-6P or fructose-6P, using for example, 1, 2, 3, 4 or 5 enzymatic steps. Nonlimiting examples of a glucose-6P or fructose-6P metabolite include glucose (typically formed from glucose-6P in a single step mediated by a phosphatase), mannose, and quercetin-3-glucoside (typically formed from glucose-6P via three enzymatic steps involving phosphoglucomutase, pyrophosphorylase, and the glucotransferase called UGT73B3). It should be understood that while the invention is illustrated by accumulating a hexose, such as glucose, as an accumulated product, other products of interest can be accumulated within the cell and the methods and cells for accumulation of hexoses can be readily extended to the accumulation of other glucose-6P or fructose-6P metabolites. More particularly, when the invention is described herein with respect to glucose, mannose, or other 6-carbon sugars, it is to be understood that analogous methods and cells can be used to produce other glucose-6P or fructose-6P metabolites. The compounds to be accumulated, insofar as they are metabolites of glucose-6P or fructose-6P, typically contain a six-carbon glucose, fructose or mannose moiety. These compounds include not only simple hexoses, but larger compounds that include hexose moieties, such as various glucosides, e.g., hyaluronic acid, and the like. Unless otherwise specified herein, the term “hexose” includes not only simple 6-carbon sugars, but also compounds that contain one or more 6-carbon sugar moieties.

Thus, in some embodiments, the metabolically engineered cell forms and accumulates a hexose. However, the invention is not limited to the formation and accumulation of a hexose such as glucose or mannose. The metabolically engineered cell can accumulate any metabolite of interest from fructose-6P or glucose-6P, using the principles described herein. Thus the invention provides a metabolically engineered cell and method for producing glucose-6P and fructose-6P metabolites, for example, glucosylated compounds produced by the metabolic pathway glucose-6P→glucose-1P→UDP-glucose→glucosylated compounds.

In some embodiments, the metabolically engineered cell includes one or more of modifications (a), (b), (c) or (d) as follows: (a) deletion or inactivation (e.g., knockout) of at least one gene encoding a gene product involved in metabolic uptake of the hexose; (b) deletion or inactivation (e.g., knockout) of at least one gene encoding a gene product involved in the primary means of metabolism of the hexose, for example at least one gene encoding a gene product involved in glycolysis; (c) deletion or inactivation (e.g., knockout) of at least one gene encoding a gene product involved in the pentose phosphate pathway; (d) overexpression of at least one phosphatase. In a preferred embodiment the metabolically engineered cell contains modification (a) and (b); in a particularly preferred embodiment, the metabolically engineered cell contains modifications (a), (b), and (c); in another embodiment, the metabolically engineered cell contains modifications (a), (b), (c), and (d). In an exemplary embodiment where the hexose is glucose, modification (a), if present, is exemplified by knockouts of the ptsG, manZ and glk genes or their counterparts; modification (b), if present, is exemplified by knockout of the pfkA gene or its counterpart; modification (c), if present, is exemplified by knockout of the zwf gene or its counterparts; and modification (d), if present, is exemplified by overexpression of an alkaline phosphatase or a haloacid dehalogenase-like phosphatase. Hexoses such as fructose can be formed and accumulated using analogous gene deletions/inactivations in the metabolic uptake of the hexose.

In some embodiments, modification (a) includes deletion or inactivation (e.g., knockout) of at least one gene encoding a gene product involved in metabolic uptake of the hexose to be accumulated, e.g., glucose, fructose, or mannose. In other embodiments, modification (a) includes deletion or inactivation (e.g., knockout) of at least one gene encoding a gene product involved in metabolic uptake of a hexose such as glucose, fructose, or mannose, while the product to be accumulated is a different hexose (i.e., compound containing a hexose moiety) such as a glucoside. Additionally or alternatively, modification (a) can optionally include deletion or inactivation (e.g., knockout) of at least one gene encoding a gene product involved in metabolic uptake of the glucoside (or other hexose moiety containing product) which is to be accumulated.

Advantageously and surprisingly, when cultured in the presence of a non-hexose carbon source, such as a pentose, sugar alcohol, or glycerol, the metabolically engineered cell forms and accumulates a hexose.

In some embodiments, the invention provides a cell that is genetically engineered to accumulate D-glucose from one or more pentose carbon sources, such as D-xylose and L-arabinose. Metabolic uptake of glucose is disrupted or prevented so that glucose can accumulate within the cell, and the formation of D-glucose by the cell is optionally enhanced by disrupting or preventing D-fructose-6P and/or D-glucose-6P metabolism. In some embodiments, the cell can synthesize and accumulate D-glucose from either D-xylose or L-arabinose as sole carbon sources.

More generally, the carbon source can be, for example, a pentose (such as xylose or arabinose) or a sugar alcohol (such as glycerol). In some embodiments, the cells are used to generate glucose or other hexoses from 1-carbon compounds.

The metabolically engineered cells of the present invention may be, for example, bacterial cells or yeast cells. Exemplary bacterial cells are Escherichia coli cells.

The present invention also provides methods for formation of a hexose comprising culturing a metabolically engineered cell in the presence of a carbon source under conditions to allow the cell to accumulate the hexose. Exemplary hexoses include glucose, mannose, and fructose. In some embodiments, the carbon source is a pentose (such as xylose or arabinose) or a sugar alcohol (such as glycerol).

In some embodiments, the methods of the invention may further include isolating or purifying the hexose. In some embodiments, the methods of the invention may further include isolating the hexose from the cell or the cell culture medium.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 shows the principal pathways involving interconversions between xylose, arabinose, fructose and glucose. The knockouts shown by double lines are for Escherichia coli

MEC143 which accumulates D-glucose: proteins involved in glucose uptake and phosphorylation ([A] ptsG manZ glk), glucose-6P dehydrogenase ([E] zwj) and 6P-fructokinase ([N], pfkA).

FIG. 2 shows the pentose phosphate pathway and upper glycolysis of Escherichia coli considering either L-arabinose or D-xylose as carbon sources and which form D-xylulose-5P as a common intermediate (dashed line). Genes (underlined) and the enzymes they code are: glucose-l-phosphatase (EC 3.1.3.10, encoded by agp), phosphoglucomutase (EC 5.4.2.2, pgm), D-glucose-P isomerase (EC 5.3.1.9; encoded by pgi), D-glucose-6P 1-dehydrogenase (EC 1.1.1.49; zwf), and 6-phosphofructokinase (EC 2.7.1.11; pfkA). E. coli ptsG manZ glk (strain ALS1048) has gene deletions which prevent the microbe from metabolizing D-glucose (knockout indicated by double line). The conversion of D-fructose-1,6P₂ to glyceraldehyde-3P is mediated by enzymes in several steps (dotted line).

FIG. 3 shows a comparison of E. coli strains for the production of D-glucose from 5 g/L L-arabinose or 5 g/L D-xylose. All strains are derived from ALS1048 (MG1655 ptsG manZ glk) and have additional gene knockouts as indicated. Strains which accumulated D-glucose were studied in 3-6 replicate cultures grown at 50 mL in a 250 mL shake flask, and error bars show the standard error of the measurements from these replicate samples. An asterisk (*) indicates a significant difference (P<0.10) in yield of D-glucose from D-xylose compared to from L-arabinose.

FIG. 4 shows confirmation of D-glucose production from xylose, using an overlay of the two-dimensional, ¹H, ¹³C-HSQC NMR spectra of a sample of product from the pentose (black contours, medium after xylose was exhausted by cells, with D₂O added to 7%) and a sample using authentic glucose-6P in medium (gray contours, with D₂O added to 7%). The molecules are distinguished because the presence of the phosphate group on D-glucose-6P promotes characteristic ¹H and ¹³C chemical shift changes, compared to D-glucose, for the nuclei at positions nearer the site of glucose attachment (positions 6, 5, and 4, with smaller changes at 3, 2 and 1). Comparison with spectra of authentic D-glucose (not shown) confirms the identities. The data demonstrate that D-glucose is the fermentation product from xylose, and that D-glucose does not form by extracellular hydrolysis of D-glucose-6P under the conditions of the experiments.

FIG. 5 shows accumulation of D-glucose (▾) and D-mannose (▴) from 20 g/L D-xylose () by E. coli MEC143 (MG1655 ptsG manZ glk pfkA zwf). Cell density is measured as optical density (□).

FIG. 6 shows glucosylation of the aglycon cyanidin using a glycosyltransferase [B].

FIG. 7 shows a proposed biochemical pathway for the formation of the D-gluconate from D-glucose. Two knockouts (double lines) in MEC143 are shown (these knockouts are also shown in FIG. 1).

FIG. 8 shows a proposed biochemical pathway for the formation of the rare sugar L-gulose from either D-xylose or D-glucose. D-sorbitol is also known as D-glucitol.

FIG. 9 shows a conversion of quercetin to quercetin-3-O-glucoside mediated by the glycotransferase UGT73B3 from Arabidopsis thaliana. UDP-glucose serves as the glucose donor.

FIG. 10 shows production of quercetin-3-O-glucoside (▴) from 30 mg/L quercetin and 4 g/L xylose (). Glucose (□) is a by-product from the conversion.

FIG. 11 shows metabolic pathways from D-xylose or L-arabinose to glucose in 5 or 6 enzymatic respective steps. Key reversible enzymes include transketolase and transaldolase. Metabolite abbreviations shown are D-xylulose-5-phosphate (Xu5P), D-ribulose-5-phosphate (Ru5P), D-ribose-5-phosphate (R5P), D-sedoheptulose-7-phosphate (S7P), D-erythrose-4P (E4P), D-fructose-6-phosphate (F6P), and D-glucose-6-phosphate (G6P). As shown in the figure, in addition to knockouts in ptsG, manZ, and glk, all strains used in this study have gene deletions in glucose-6P 1-dehydrogenase (first enzyme mediating entrance of G6P into pentose phosphate pathway coded by zwf gene) and 6-phosphofructokinase (first enzyme mediating conversion of F6P to Gly3P coded by pfkA gene).

FIG. 12 shows mass glucose yield from xylose or arabinose (g/g) during batch growth of various phosphatase knockout strains of Escherichia coli.

FIG. 13 shows a comparison of mass glucose yield from xylose or arabinose (g/g) using E. coli MEC143 during carbon-limited, batch or phosphate-limited growth.

### DETAILED DESCRIPTION OF ILLUSTRATIVE EMBODIMENTS

The invention provides microorganisms, particularly bacteria, such as E. coli cells, that have been metabolically engineered to accumulate a six carbon sugar, such as glucose and mannose, or other glucose-6P or fructose-6P metabolites. The hexose or other metabolite is typically accumulated within the cell, although in some embodiments the hexose is secreted into the medium.

The basic metabolic engineering strategy for accumulating a hexose relies on two principles: disabling the metabolic uptake system for the hexose to be accumulated, and disabling the primary catabolic routes for the utilization of the phosphorylated form of the hexose to be accumulated. In other words, the cell is metabolically engineered to prevent the cell from consuming the compound to be accumulated (i.e., the hexose), and further to permit accumulation of the sugar-phosphate intermediate as a step toward accumulating the product sugar. Thus, to disable catabolism and thereby permit the unnatural accumulation of a hexose as a final product, the microorganism is metabolically engineered according to the invention to block glycolysis, and/or block metabolic re-entry of the hexose into the pentose phosphate pathway. Preferably, both glycolysis and metabolic re-entry of the hexose into the pentose phosphate pathway are blocked. These metabolic changes effectively divert carbon flow toward the hexose. For example, a strategy that allowed accumulation of glucose in the exemplary organism MEC143 can be summarized as 1) the native glucose uptake system (ptsG manZ glk) was eliminated and 2) major catabolic routes for glucose (pfkA and secondarily zwf) were blocked.

Advantageously, the metabolically engineered cells of the invention can accumulate a six carbon sugar as a final product when supplied with a five carbon sugar (“pentose”) such as xylose or arabinose as a carbon source. Essentially, the five carbon sugar is converted to a six carbon sugar. Alternatively or additionally, the metabolically engineered cells of the invention can accumulate a six carbon sugar as a final product when supplied with glycerol as a carbon source.

Microbial accumulation of a hexose as a “final product” means that the metabolically engineered microorganism exhibits hexose levels that are increased relative to a microorganism that has not been metabolically engineered. Formation of hexoses in microbial cells has the inherent advantages of high volumetric rates; for example, 0.4 g/lh of glucose was observed. Hexose is accumulated in a measurable amount; preferably the microbial cells of the invention accumulate hexose at a level between about 0.10 g/g-0.25 g/g with a pentose as carbon source. The hexose produced by the cells described herein may be isolated and, optionally, purified. The hexoses can be isolated directly from the cells, or from the culture medium, for example, during an aerobic or anaerobic growth process. Isolation and/or purification of a hexose or a hexose derived product can be accomplished using known methods.

It should be noted that to our knowledge, no previous research has demonstrated bacterial glucose production. However, numerous reports have described research on related saccharide products. For example, Acetobacter xylinum has been coaxed to accumulate cellulose (Nakai et al., 1999, Proc Natl Acad Sci 96, 14-18), and Corynebacterium glutamicum overexpressing the glgC gene encoding ADP-glucose pyrophosphorylase accumulates 90 mg glycogen/g dry cell weight (Seibold et al., 2007, Microbiol. 153, 1275-1285). A small amount of glycogen formation has even been accomplished in E. coli by overexpressing glgC although the goal was to slow acetate formation rather than form glycogen (Dedhia et al., 1994, Biotechnol Bioeng 44, 132-139).

Surprisingly, the metabolically engineered cells of the invention, which have been rendered unable to metabolize certain sugars, are nonetheless able still grow and function biologically. The invention thereby affords the opportunity to direct these hexoses into valuable products such as the so-called “rare” (unnatural) sugars, and likewise into a wide variety of glycosylated (glucuro-, gluco-, galacto-, manno-derivatized) compounds. Furthermore, this invention serves as a means of building onto a carbon backbone from for example, 5-carbon to 6-carbon length. Bacterial hexose formation and accumulation according to the invention thereby provides the basis for and makes possible sugar conversions, the accumulation of sugar-derived products, and one-carbon fixation in bacteria such as E. coli.

The ability to generate hexoses such as glucose as products of controlled bioprocesses opens up a wide range of unexplored research terrain. For example, as noted, the present invention a means to generate glucose from 1-carbon compounds. The incorporation of methanol into glucose can be accomplished using key enzymes from the ribulose monophosphate pathway, however other 1C sources such as CO₂ or methane could feasibly be converted into monosaccharide products. The invention provides the foundation for many other avenues of future research in E. coli and other organisms which may have unique abilities to utilize 1-carbon compounds.

The present invention also provides a platform to generate unique carbohydrate products which, in the absence of a means to direct carbon to glucose, could not otherwise be effectively produced. Typically, sugars such as glucose are immediately phosphorylated and metabolized and therefore their intracellular concentrations are vanishingly minute. This invention makes possible the formation of many other products which would not have been feasible without the intracellular accumulation of the sugar. The efficient production of literally thousands of glycosylated products, many with pharmaceutical applications and for further research purposes, can be facilitated by this invention. Similarly, optically pure rare sugars are increasingly important in biochemical research, including the development of new pharmaceutical therapies. Such monosaccharides are involved in cell recognition, signaling, and the development of diseased states (Allen et al., 2001, J. Amer. Chem. Soc. 123, 1890-1897; Koeller and Wong 2000, Nat Biotechnol 18, 835-841; Bartolozzi and Seeberger, 2001, Curr Opin Struct Biol 11, 587-592).

A preferred product of the present invention is a 6-carbon monosaccharide having the chemical formula C₆H₁₂O₆, which can be referred to as a hexose, although the term hexose is used more broadly herein to include other, 6-carbon sugar moiety containing compounds as well. The hexose may be in the D configuration, the L configuration, or a combination thereof. Hexoses are typically classified by functional group. For example, aldohexoses have an aldehyde at position 1 and include, without limitation, allose, altrose, glucose, mannose, gulose, idose, galactose, and talose, and ketohexoses have a ketone at position 2 and include, without limitation, psicose, fructore, sorbose, and tagatose. A hexose also contains 6 hydroxyl groups and the aldehyde or ketone functional group in the hexose may react with neighbouring hydroxyl functional groups to form intramolecular hemiacetals or hemiketals, respectively. The resulting ring structure is related to pyran, and is termed a pyranose. The ring spontaneously opens and closes, allowing rotation to occur about the bond between the carbonyl group and the neighbouring carbon atom, yielding two distinct configurations (α and β). The hexose may be in either the a configuration or the _{R} configuration. The hexose of the invention may be either a linear or a ring structure. Exemplary hexoses produced are glucose and mannose. The presence of the accumulating hexose can be detected using standards techniques, such as GC-MS.

The present invention encompasses not only metabolically engineered cells that can form and accumulate a hexose, but also methods for making said cells and methods for making and isolating a hexose and, optionally, hexose metabolites, derivatives or hexose-containing molecules or products, from said cells or cell culture. Examples of such metabolically engineered cells, such as those that are engineered to express a modified pentose phosphate pathway, as well as methods for making said cells and methods for making and isolating a hexose and, optionally, its metabolites and derivatives are described in more detail below.

Hexose formation and accumulation relies on preventing a specific sugar uptake system and/or sugar transport mechanism. Preventing a microbial cell from taking up a specific saccharide, including hexoses, has been demonstrated (see for example, U.S. Patent Application Publication No. 2010/0129883, which is incorporated by reference herein). However, microbial cells also have catabolic routes for the utilization of sugars and will re-use, rather than accumulate the desired hexose. Therefore, hexose formation and accumulation also relies on diverting carbon to that unmetabolizable sugar. Therefore, modification of the PP pathway in the instant invention to accomplish the unnatural accumulation of hexoses includes both 1) preventing uptake and/or transport of the desired hexose, and 2) diverting carbon to the desired hexose.

Modification of the PP pathway and associated genes are described below with reference to Escherichia coli genes, as a preferred microbial cell. However, it should be understood that a comparable gene (homolog or ortholog) in another microbial cell may use a different nomenclature but can nevertheless be used or targeted similarly to the analogous E. coli gene.

Metabolic Engineering to Prevent Uptake and/or Transport of the Desired Hexose

A metabolically engineered cell of the invention is one that is engineered to disrupt or prevent metabolic uptake of a hexose to be accumulated. “Metabolic uptake” can include, without limitation, processes involved in transmembrane transport into or within a cell, and/or intracellular utilization or metabolism of the hexose. For example, a compound that typically serves as a cell nutrient (e.g., a hexose such as glucose) is normally metabolically converted by the cell into one or more compounds that are generated to allow the cell to grow and perform various functions. Disrupting the metabolic “uptake” of a nutrient, such as a hexose, prevents the hexose from being converted into one or more of these secondary compounds, thereby preventing utilization of the hexose for various intracellular purposes. In order to disrupt metabolic uptake of the desired hexose, all or a portion of the uptake system for that hexose may be eliminated. A number of genes involved in the uptake of hexoses are known in the art. For example, genes involved in glucose uptake include, without limitation, glucokinase (glk), glucosephosphotransferase enzyme II (ptsG), mannose PTS protein IIA(III) (manX), pel protein (manY),and mannosephosphotransferase enzyme IIB (manZ). Such a strain will not be able to take up mannose, either. Preferably, glucose consumption is prevented by knock-out of three principal genes involved in glucose uptake, ptsG; manZ, manY or manX; and glk. Therefore, a cell that cannot take up glucose (and is thus useful for accumulating glucose) can be created, for example, by disrupting the following three genes: ptsG; manZ, manY or manX; and glk.

Genes involved in mannose uptake include, without limitation, mannose PTS protein IIA(III) (manX), pel protein (manY), and mannosephosphotransferase enzyme IIB (manZ). Therefore, cell that cannot take up mannose (and is thus useful for accumulating mannose) can be created, for example, by disrupting at least one of the following genes: manX, manY or manZ.

Genes involved in galactose uptake include, without limitation, galactose binding protein (mglB), galactose transport membrane protein (mglC), galactose ATPase protein (mglA), and galactokinase (galK). Therefore, cell that cannot take up galactose (and is thus useful for accumulating galactose) can be created, for example, by disrupting at least one of the following genes: mglB, mglC, mglA, or galK.

In a preferred embodiment, the microbial cell of the invention is engineered to prevent glucose consumption by knock-out of ptsG manZ, and glk.

Advantageously, the microbial cells of the invention, although unable to take up the desired hexose, can still grow and function biologically thus enabling the accumulation of the desired hexose.

Similarly, in order to prevent transport of the desired hexose, the transport system for that hexose may be eliminated. A number of genes involved in hexose transport are known in the art. For example, genes involved in glucose transport include, without limitation, galactose permease (galP) and the putative transporter encoded by the yih() gene.

### Metabolic Engineering to Divert Carbon to the Desired Hexose

Diverting carbon flow to the desired hexose can be achieved by blocking glycolysis and/or blocking metabolic re-entry of the hexose or its intermediates into the pentose phosphate pathway. Preferably, both glycolysis and metabolic re-entry of the hexose are blocked.

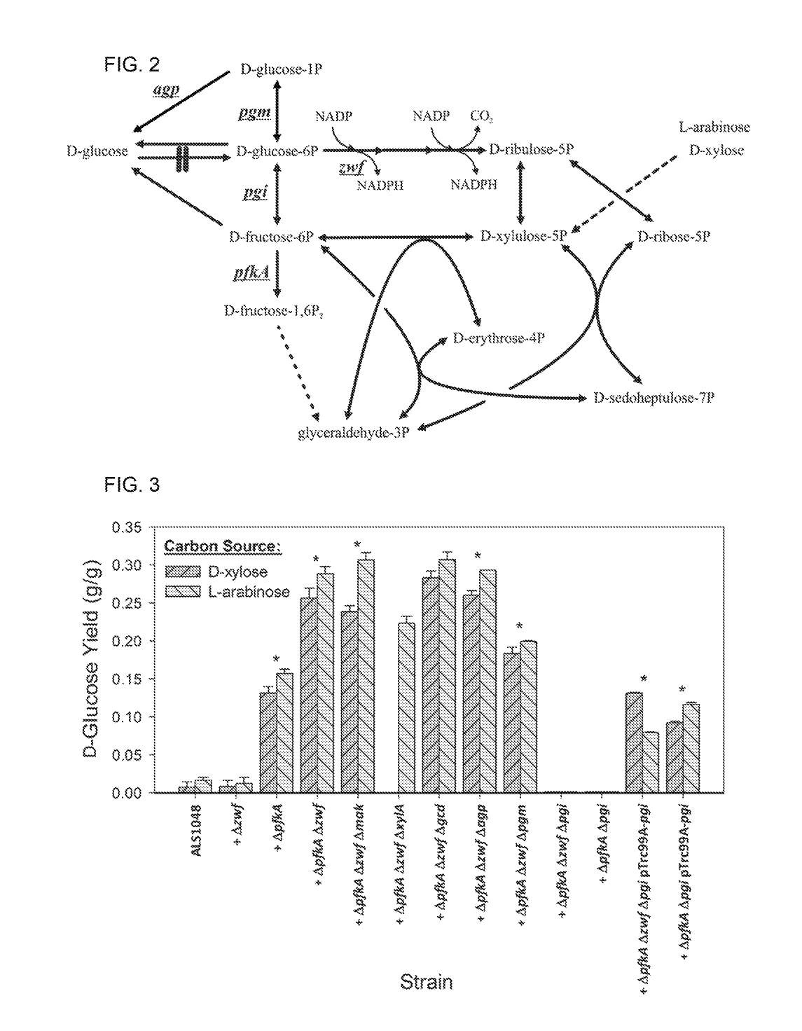

Blocking Glycolysis. Glycolysis is the metabolic pathway that converts glucose into pyruvate and is a purely anaerobic reaction. In a cell without a complete glycolytic pathway, however, the metabolism of a pentose becomes a branched pathway with two separate products, D-fructose-6P and D-glyceraldehyde-3P (as represented in FIG. 2). Prevention of the glycolytic conversion of D-fructose-6P to D-glyceraldehyde-3P leaves D-fructose-6P available for the formation of hexoses. Advantageously, D-glyceraldehyde-3P then remains available for the generation of ATP, NADH, and the precursors which exist metabolically “below” D-glyceraldehyde-3P via the terminal steps of glycolysis and the tricarboxylic acid cycle.

The pentose phosphate (PP) pathway is a complex metabolic pathway that facilitates the interconversion of phospho-sugars between 3 and 7 carbons in length. In organisms having the requisite kinases and/or sugar transport mechanisms, the PP pathway also provides numerous convenient entry points for the catabolism of a wide range of sugars including D-xylose and L-arabinose.

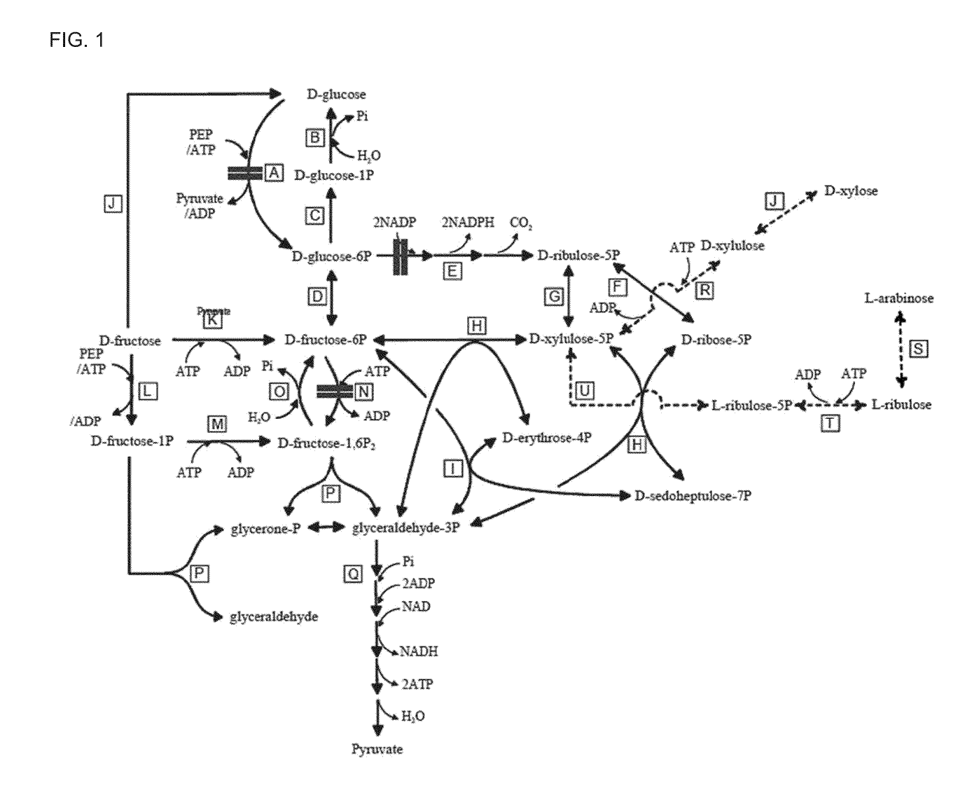

In the present invention, the engineered accumulation of a hexose involves the modification of one or more aspects of the pentose phosphate (PP) pathway. Exemplary modifications are shown in FIG. 1, which shows knockouts of proteins involved in glucose uptake and phosphorylation including ([A] ptsG manZ glk), glucose-6P dehydrogenase ([E] zwf) and 6P-fructokinase ([N], pfkA).

The modified PP pathway of the invention thus includes disruption of glycolysis between D-fructose-6P and D-glyceraldehyde-3P (as shown by dotted line in FIG. 2). A gene involved in the conversion of D-fructose-6P to D-glyceraldehyde-3P is, without limitation, 6P-fructokinase, which is encoded by the pfkA gene. Therefore, a cell having blocked glycolysis of D-fructose-6P can be created, for example, by disrupting the pfkA gene.

Blocking Metabolic Re-Entry. Metabolic re-entry of the desired hexose into the pentose phosphate pathway can be blocked by eliminating the primary catabolic routes for the utilization of the desired hexose. Prevention of the conversion of D-glucose-6P to D-ribulose-5P forces the intermediate to remain as D-glucose-6P, and advantageously, leaves D- glucose -6P available for the formation of hexoses.

The modified PP pathway of the invention thus prevents re-entry of the desired hexose into the PP pathway. Preferably, the modified PP pathway of the invention prevents re-entry of glucose by eliminating D-fructose-6P. As shown in FIG. 2, enzymes which enable D-glucose-6P to convert to D-ribulose-5P and whose elimination blocks metabolic re-entry of glucose include, for example, D-glucose-6P 1-dehydrogenase (encoded by the zwf gene), 6P-gluconolactonase (encoded by the pgl gene), and 6P-gluconate dehydrogenase decarboxylating (encoded by the gnd gene). Preferably, the enzyme eliminated in the modified pathway is D-glucose-6P 1-dehydrogenase (DO. Therefore, a cell that cannot metabolize glucose through the

PP pathway can be created, for example, by disrupting the zwf gene.

Elimination of the pfkA gene (encoding 6P-fructokinase) and the zwf gene (encoding glucose-6P 1-dehydrogenase) can be analyzed together by the following stoichiometric equations for xylose uptake (Eq. 1), noting 1 ATP/xylose is believed necessary for transport via the ABC system (Linton, 1996):

3 xylose+6 ATP→3 xylulose-5P+6 ADP [1]

for pentose phosphate interconversions (Eq. 2):

3 xylulose-5P→glyceraldehyde-3P+2 fructose-6P [2]

and for glucose formation (Eq. 3):

2 fructose-6P+2 H₂O→2 glucose+2 Pi [3]

to yield a net theoretical reaction for glucose formation from xylose (Eq. 4):

3 xylose+6 ATP+2 H₂O→2 glucose+glyceraldehyde-3P+6 ADP+2 Pi [4]

The assimilation of one mole of glyceraldehyde-3P generates 3 CO₂, 5 NADH, 1 NADPH, 1 FADH, and 3 ATP, sufficient to supply biosynthetic needs. The maximum theoretical yield of glucose from xylose according to Eq. 4 is therefore 0.67 mol glucose/mol xylose or 0.80 g/g. E. coli can convert xylose to glucose and accumulate gram quantities of glucose as a final product, as shown in Example 1 and in the table below.

| | | Glucose formed (g/L) |
|---|---|---|
| Strain | Genotype | from xylose (5 g/L) |
| ALS1048 | MG1655 ptsG manZ glk | 0 |
| MEC132 | MG1655 ptsG manZ glk pfkA | 0.6 |
| MEC144 | MG1655 ptsG manZ glk zwf | 0 |
| MEC143 | MG1655 ptsG manZ glk zwf pfkA | 1.4 |

Therefore, a cell having blocked metabolic re-entry of the accumulating hexose can be created, for example, by disrupting the pfkA gene and the zwf gene.

In a preferred embodiment, the microbial cell of the invention is engineered to divert carbon to glucose by knock-out of both pfkA and zwf.

The PP pathway discussed principally focuses on the conversion of xylose to glucose via the PP pathway. However, it should be understood that synthesis and accumulation of any desired hexose may be achieved with modifications to the appropriate synthetic pathway that 1) prevent uptake of the desired hexose, and 2) divert carbon to the desired hexose.

### Enhancing Glucose Accumulation

The final step in the conversion of pentoses via fructose-6P to glucose is the dephosphorylation of glucose-6P:

glucose-6P→glucose+Pi [6]

Dephosphorylation is mediated by phosphatases, enzymes which typically act on multiple substrates. Overexpression of one or more phosphatases can result in increased yields of glucose. Thus, the metabolically engineered cell optionally overexpresses at least one phosphatase. The phosphatase that is overexpressed can be endogenous to the cell, or it can be a heterologous phosphatase (i.e., one not expressed in a wild-type cell). A vector, such as a plasmid or cosmid, the operably encodes the phosphatase can be introduced into the cell, or a polynucleotide operably encoding the phosphatase can be genomically integrated into the cell using techniques known to the art. The phosphatase can be, for example, an alkaline phosphatase, a haloacid dehalogenase-like phosphatase, or any phosphatase having measurable activity on glucose-6P. Exemplary phosphatases include, without limitation, phoA, ybiV, yfbT, yniC yidA or yigL.

More generally, overexpression of one or more phosphatases can be used to enhance the production of any hexose from its phosphorylated counterpart, provided that the host cell is engineered to eliminate metabolism of hexose.

Additionally, it is expected that if a sugar other than glucose (and/or its derivatives) is desired from glucose-6-phosphate or fructose-6-phosphate, certain phosphatases could be knocked out and others could be overexpressed in order to direct the cell toward accumulation of the other sugar. Representative examples include the following:
1) D-fructose-6P 4 D-fructose overexpress yfbT or yidA and knockout pgi and/or knockout phosphatases involved in glucose-6P dephosphorylation. YfbT and YidA are phosphatases with identified activity on fructose-6P (Kuzentsova et al., J Biol Chem. 2006 Nov. 24; 281(47):36149-61);
2) D-glucose-6P 4 D-glucono-1,5-lactone-6P 4 D-gluconate-6P 4 D-gluconate overexpress yjjG, which codes a phosphatase with identified activity on D-gluconate-6P (Kuzentsova et al., J Biol Chem. 2006 Nov. 24; 281(47):36149-61);
3) D-fructose-6P 4 D-mannose-6P 4 D-mannose overexpress yniC, which codes a phosphatase with identified activity on D-mannose-6P (Kuzentsova et al., J Biol Chem. 2006 Nov. 24; 281(47):36149-61); and
4) D-fructose-6P 4 D-mannitol-1P 4 D-mannitol overexpress yniC, which codes a phosphatase with identified activity on D-mannose-6P ((Kuzentsova et al., J Biol Chem. 2006 Nov. 24; 281(47):36149-61).

Additionally, it can be seen from Eq. 6 that, by operation of the law of mass action, culturing a metabolically engineered cell of the invention under phosphate limited conditions can drive the reaction toward glucose and enhance its accumulation via that mechanism as well. Thus, in the method of the invention, the metabolically engineered is optionally cultured under phosphate-limited conditions. Preferably, a chemostat culture is utilized. Growth under phosphate-“limited” conditions means that the cells are growing at a rate slower than the maximum growth rate they exhibit when supplied with a non-limiting amount of phosphate (i.e., when supplied with phosphate at a level where the growth rate no longer increases with increasing amounts of phosphate). One of skill in the art can readily determine a limiting concentration of phosphate (or any nutrient) for a particular cell culture, as there is a direct relationship between growth rate and concentration of the limiting nutrient. For example, the concentration of a nutrient which corresponds with “limiting conditions” can be modeled using a “Saturation Constant” also known as the “Monod Constant”. One interpretation of the Monod Constant is that it is the concentration of limiting nutrient that will result in the cells growing a one-half their maximum specific growth rate. Thus, if a bacterial culture has a maximum specific growth rate of 0.80 h-1 (exemplary for E. coli growing using xylose as a carbon source), and the growth rate is limited to 0.40 h-1, the concentration of the limiting nutrient in the culture will be considered to be the Monod constant. An exemplary Monod constant can range between about 1-10 mg/L. Other models known to the art can also be used to determine the relationship between limiting nutrient concentration and growth rate. For example, Shehata et al. (“Effect of Nutrient Concentration on the Growth of Escherichia coli” J. Bacteriol. 107(1):210-216; 1971) show that a phosphate concentration of about 0.08 mM (8 mg/L) results in a growth rate of 0.4 h-1, which is about half of maximum they reported.

### Carbon Source

A preferred carbon source for use in the present invention is a pentose. As used herein a “pentose” is any monosaccharide with five carbon atoms, having the chemical formula C₅H₁₀O₅. The pentose may be in the D configuration, the L configuration, or a combination thereof. Pentoses are typically classified by functional group. For example, aldopentoses have an aldehyde functional group at position 1 and include, without limitation, arabinose, lyxose, ribose, and xylose, and ketopentoses have a ketone functional group in position 2 or 3 and include, without limitation, ribulose and xylulose. A pentose also contains 5 hydroxyl groups and the aldehyde or ketone functional group in the pentose may react with neighbouring hydroxyl functional groups to form intramolecular hemiacetals or hemiketals, respectively. The resulting ring structure is related to furan, and is termed a furanose. The ring spontaneously opens and closes, allowing rotation to occur about the bond between the carbonyl group and the neighbouring carbon atom—yielding two distinct configurations (α and β). The pentose may be in either the a configuration or the β configuration. The pentose of the invention may be either a linear or a ring structure. Preferably, the carbon source of the present invention is xylose or arabinose.

Also useful as a carbon source in the pathway of the instant invention are sugar alcohols having the chemical formula H(HCHO)ₙ₊₁H. A sugar alcohol (also known as a polyol, polyhydric alcohol, polyalcohol, or glycitol) is a hydrogenated form of a sugar, whose carbonyl group (aldehyde or ketone) has been reduced to a primary or secondary hydroxyl group.

Preferably, the sugar alcohol of the invention has at least 3 carbons. For example, a 3-carbon sugar alcohol is a glycerol. Non-limiting examples of 4-carbon sugar alcohols are erythritol and threitol. Non-limiting examples of 5-carbon sugar alcohols include arabitol, xylitol, ribitol, and glycerol. Non-limiting examples of 6-carbon sugar alcohols include mannitol, sorbitol, galactitol, fucitol, iditol, and inositol. Preferably, the sugar alcohol is glycerol.

While the embodiments described herein are typically directed to using a “pentose” as a carbon source, it should be understood that sugar alcohols may also be substituted as the carbon source for any embodiment.

Additionally or alternatively, the present invention allows utilization of 1-carbon compounds as a carbon source, for example as means of building on to a carbon backbone, as described in more detail elsewhere herein.

### Host Cells

The present invention provides microbial cells that are metabolically engineered for formation and accumulation of hexoses, as well methods for making said cells and methods for producing and isolating hexoses and, optionally, its derivatives and metabolites, from said cells or cell culture. The microbial cell of the invention is engineered to produce hexoses using standard genetic engineering techniques. Preferably, the microbial cell of the invention natively contains a pentose phosphate (PP) pathway. The cell can be a eukaryotic cell or a prokaryotic cell. Preferably, the cell is a prokaryotic cell such as a bacterial cell; however single cell eukaryotes such as protists or yeasts are also useful as microbial cells of the invention. Microbial cells can be individually engineered to produce hexoses as described herein.

The term “microbe” is used interchangeably with the term “microorganism” and means any microscopic organism existing as a single cell (unicellular), cell clusters, or multicellular relatively complex organisms. Microbial cells include, for example, bacteria, yeast, algae, protozoa, microscopic plants such as green algae, and microscopic animals such as rotifers and planarians. Preferably, a microbial host used in the present invention is single-celled.

The cell of the invention can be genetically engineered through any technique known in the field. For example, the cell may be engineered for enhanced gene expression, reduced/eliminated gene expression, or altered gene expression. Engineering techniques may include the introduction of polynucleotides, directed mutagenesis, or gene disruptions including mutagenesis, gene deletion, gene inactivation, or “knock-out,” and heterologous gene transformation. Such methods are well known in the art; see, e.g., Sambrook et al, Molecular Cloning: A Laboratory Manual., Cold Spring Harbor Laboratory Press (1989), and Methods for General and Molecular Bacteriology, (eds. Gerhardt et al.) American Society for Microbiology, chapters 13-14 and 16-18 (1994).

Microbial cells useful in the invention can include, without limitation, bacteria, protists and fungi, including yeasts; preferably, the microbial cell includes a metabolic pathway for glycolysis and optionally a pentose phosphate (PP) pathway. See, e.g., Sanchez-Riera, “Production of Organic Acids” in Biotechnology, Vol. V, UNESCO-EOLSS, Encyclopedia of Life Support Systems. Example of suitable bacteria include Lactobacillus species, such as L. delbrueckii, L. leichmannii, L. bulgaricus, L. pentosus, L. casei, L. lactis, L. plantarum, and L. pentosus; Actinobacillus species such as A. succinogenes; Acetobacter species such as A. pasteurianus; Gluconobacter species, such as G. oxydans; Escherichia species such as E. coli; Clostridium species, such as C. stercorarium; and Corynebacterium species such as C. glutamicum. Examples of suitable fungi include Aspergillus species such as A. niger, A. nidulans, A. wentii, A. tereus, A. flavus, A. carbonarius, A. aculeatus, A. ficuum, A. awamori, A. oryzae, A. candidus, and A. itaconicus; Candida species such as C. tropicalis, C. oleophila, C. guilliermondii, and C. citroformans; Saccharomyces cerivisae; Yarrowia lipolytica; Rhizopus oryzae; Penicillium species such as P. isariiforme and P. chrysogenum; Neurospora species, such as N. crassa; Gibberella species such as G. fujikuroi; Talaromyces species such as T. stipatatus; and Pichia species such as P. stipitis and P. anomala.

E. coli is an exemplary illustrative organism for the formation and accumulation of hexoses, and the genes that are metabolically engineered are described primarily with reference to E. coli genes. Bacteria such as E. coli are ideal for the synthesis of hexoses because of their fast growth and substrate utilization, and because of the availability of genetic tools which ease metabolic manipulation. However, the invention is not intended to be limited to embodiments that utilize E. coli. It is to be understood the metabolic engineering described herein with reference to E. coli can be readily adapted to many other microbial cells, by manipulating counterpart genes in a similar fashion. Counterpart or comparable genes (e.g., homologs or orthologs) in other microbes may use a different nomenclature but can nevertheless be utilized, altered, or targeted similarly to the analogous E. coli gene. Counterpart genes in other microbes, including bacteria, protists, and fungi, are well-known in the art and readily identifiable by a skilled artworker.

### Generating Metabolically Engineered Cells

The metabolically engineered microbial cell accumulates a hexose such as glucose or mannose when supplied with a pentose such as xylose or arabinose or when supplied with a sugar alcohol, such as glycerol. The molecular targets of metabolic engineering that will yield a suitable cell for use in the method of the invention naturally depend on the nature of the organism, as well as the pentose whose metabolism is to be modified. The metabolically engineered cell of the invention is modified to both 1) prevent uptake of the desired hexose, and 2) divert carbon to the desired hexose.

Particularly preferred targets for metabolically engineering cells useful in the method of the present invention are molecules involved in the PP pathway (see, for example, FIG. 1). The PP pathway can be modified by, for example, affecting the production or activity of one or more enzymes required, either directly or indirectly, to convert the carbon source into the desired hexose.

Preferably, the targeted branch of the PP is completely disrupted (e.g., via a knock-out of an essential gene). To “knock-out” a gene means to delete the gene or otherwise prevent its ability to express a functional enzyme, such that the enzyme itself or product of the specific enzymatic conversion is not detected at a measurable level. As used herein, the terms “knock-out”, “deletion”, and “inactivation” with reference to a particular gene are used interchangeably. The knockout of one or more enzymes required, either directly or indirectly, to convert the carbon source into the desired hexose can be accomplished by modifying or deleting one or more genes encoding the required enzyme(s).

Methods of disrupting or altering one or more enzymes in bacteria, plants, and animals to reduce or eliminate a cell's ability to express a functional enzyme are routine and well known in the art. Once a particular enzyme involved in the PP pathway has been identified, disruption of enzyme function can be effected at any level of gene expression (e.g., DNA replication, transcription or translation), or post-translationally. For example, enzymatic function can be inhibited when the enzyme is targeted by a molecular inhibitor, such as an antibody or a small molecule inhibitor. Translation of an RNA message into an enzyme can be disrupted, for example, by introducing a small interfering RNA, a short-hairpin RNA, or a hybridization probe into the bacteria, plant, or animal cell. Transcription of a gene encoding an enzyme can be disrupted, for example, by targeting the gene with a molecular inhibitor or physically altering the gene to prevent or confound gene replication or transcription. Cells can be metabolically engineered through the introduction of polynucleotides, as well as the directed mutagenesis of coding regions. Common gene disruption techniques include mutagenesis, gene deletion or knock-out, and heterologous gene transformation. Such methods are well known in the art; see, e.g., Sambrook et al, Molecular Cloning: A Laboratory Manual., Cold Spring Harbor Laboratory Press (1989), and Methods for General and Molecular Bacteriology, (eds. Gerhardt et al.) American Society for Microbiology, chapters 13-14 and 16-18 (1994).

A particularly useful method for engineering a metabolically engineered cell of the invention is to delete or knock out an essential gene in the PP pathway. Exemplary gene targets are discussed elsewhere in more detail and exemplary cells are set forth in Example 1.

Determination of whether enzyme activity has been reduced or eliminated can easily be made by a person of skill in the art using any basic in vitro or in vivo enzyme assay. The metabolically engineered cell of the invention will yield reduced or eliminated activity when compared with a wild-type cell in such an assay. Preferably, the cell will have no detectable enzyme activity when compared with a wild-type cell. Additionally, or alternatively, the amount of enzyme can be quantified and compared by obtaining protein extracts from the metabolically engineered cell and a comparable wild-type cell and subjecting the extracts to any of number of protein quantification techniques which are well known in the art. Methods of protein quantification may include, without limitation, SDS-PAGE in combination with western blotting and mass spectrometry.

In a particularly preferred embodiment, the metabolically engineered cell of the invention is an Escherichia coli cell which is engineered to prevent glucose consumption by knock-out of ptsG manZ, and glk and is further engineered to divert carbon to glucose by knock-out of both pfkA and zwf. Optionally the cell is further metabolically engineered to overexpress a phosphatase.

In some embodiments, the metabolically engineered cell of the invention may be further engineered to enhance hexose formation. For example, genes encoding enzymes hypothesized to be relevant to glucose formation such as the mak gene encoding mannose kinase (FIG. 1 [K]), the xylA gene encoding xylose isomerase which is also a glucose isomerase (FIG. 1 [J]), the agp gene encoding glucose 1-phosphatase (FIG. 1[B]), the pgm gene encoding phosphoglucomutase, the fructose PTS system encoded by the levF levG fruA operon, and transaldolases encoded by talA and/or talB may be knocked out. Additionally, or alternatively, to improve glucose formation, additional glucose-metabolizing genes, including the gcd gene encoding glucose dehydrogenase and genes of the Entner-Doudoroff pathway, may be deleted.

In some embodiments, the metabolically engineered cell of the invention may be further engineered to prevent the accumulating hexose from being exported from the cell. Presumably knocking out a key exporter will lead to an increased intracellular hexose and/or curtailment of growth. For example, permeases and/or transporters including galP (encoding galactose permease) and the putative transporter encoded by the yih() gene may be deleted. Alternatively, the cell of the invention may be further engineered to enhance the accumulating hexose being exported from the cell. Overexpression of identified transport proteins may enhance the extracellular accumulation of glucose.

The cells of the invention may be referred to as “genetically engineered cells” or, when the genetic engineering modifies or alters one or more particular metabolic pathways so as to cause a change in metabolism, as “metabolically engineered” cells. The goal of metabolic engineering is to improve the rate and conversion of a substrate into a desired product. In the instant invention, various aspects of the PP pathway are modified in order to optimize the rated and conversion of pentoses to hexoses.

### Cell Culture

The invention includes methods for making and isolating products such as a hexose and, optionally, hexose metabolites, derivatives or hexose-containing molecules or other products, from the metabolically engineered cells or cell culture. The products can accumulate intracellularly, or they can be secreted into the cell culture medium. The cells of the invention can be cultured aerobically or anaerobically, or in a multiple phase fermentation that makes use of periods of anaerobic and aerobic fermentation. Preferably, the cells are cultured aerobically. Batch fermentation, continuous fermentation, or any other fermentation method may be used. In some instances, cell of the invention may be supplemented with additional nutrients.

For example, the cell of the invention may be supplemented with additional carbon sources such as glycerol or acetate.

In some instances, the cell of the invention is grown in a chemostat. Preferably, the chemostat may be used to provide nutrient limited conditions. For example, the cell of the invention may be cultured in nitrogen-limited conditions, or in phosphorus-limited conditions.

Hexose yield depends on the hexose being accumulated, and in some cases on the pentose carbon source. Culturing the metabolically engineered cell of the invention can result in yields of hexose relative to a carbon source of at least 0.001 g/g, 0.05 g/g, 0.01 g/g, 0.03 g/g, 0.05 g/g, 0.07g/g, or 0.1 g/g. For example, yields of glucose relative to xylose or arabinose as a carbon source, i.e., glucose (g)/xylose/arabinose(g), can be at least 0.1 g/g, at least 0.2 g/g, or at least 0.3 g/g; yields of more rare hexoses, such as mannose, relative to a pentose carbon source can be at least 0.001 g/g, 0.05 g/g, 0.01 g/g, 0.03 g/g, 0.05 g/g, 0.07g/g, or 0.1 g/g. Yields of glucosides and other metabolites relative to a carbon source may be lower but still at a useful level, and can be, for example, at least 0.0001g/g, 0.005 g/g, 0.001 g/g, 0.05 g/g, 0.01 g/g, 0.03 g/g, 0.05 g/g, 0.07g/g, or 0.1 g/g.

Formation of Products Derived from Hexoses

Monosaccharides such as glucose or fructose likely may accumulate intracellularly to significant concentrations. The availability of such sugars intracellularly makes possible transformative research: monosaccharide-derived products could accumulate at rates and concentrations which would not have previously been deemed feasible. The invention thus further includes microbial formation of products which are derived from glucose and other hexoses.

In one aspect of the invention, the hexose-derived product is a glycosylated compound. Glycosyltransferases (GTs) are the class of enzymes which catalyze the transfer of a sugar moiety from a donor to an acceptor, aglycon molecule. Nearly 100,000 known or putative GT sequences exist in the carbohydrate-active enzyme data base (CAZy, available on the World Wide Web at cazy.org/glycosyltransferases.html). GTs are involved in the biosynthesis of glycolipids, polysaccharides, glycoproteins and numerous glycosides. Although the typical glycotransfer occurs to the nucleophilic oxygen of a hydroxyl group (O-glycosylation), substitutions can occur to other nucleophiles (N-, S- and C-glycosylation). Familiar and diverse examples of glucosides include hyaluronic acid (Yu and Stephanopoulos, 2008, Metab Eng. 10(1):24-32), as well as linamarin and lotaustralin, two cyanogenic glucosides biosynthesized from L-valine and L-isoleucine in cassava (Manihot esculenta) and broken down by β-glucosidases and α-hydroxynitrile lyases during cell rupture, resulting in the release of hydrogen cyanide (Morant et al., 2008, Phytochem.69(9):1795-813): for this reason, cassava-derived food must be processed to remove cyanide before consumption. In addition to these cyanogenic glucosides, numerous other glucosides are found in nature including alkyl and benzyl derivatives, terpenoids and flavonoids. GTs play an important role in pharmacogenetics and in the metabolism of xenobiotics (Foti and Fisher, Encyclopedia of Drug Metabolism and Interactions, 2012).

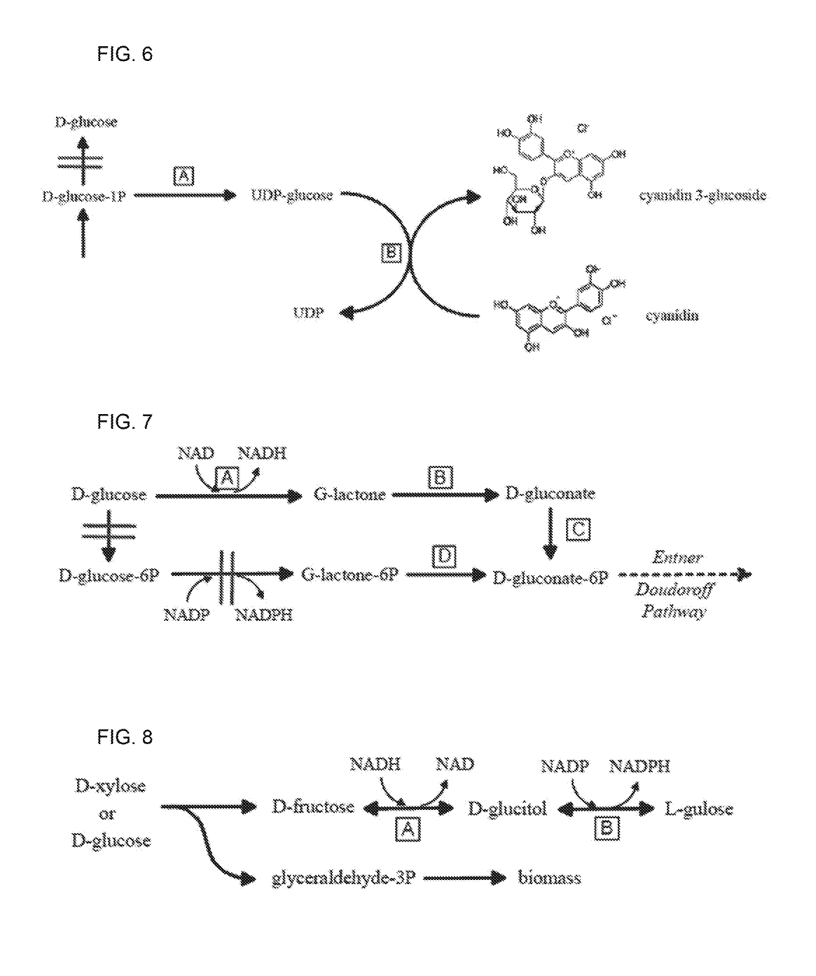

Although a wide range of acceptor substrates exist for GTs, donor substrates are almost always glyconucleotides such as uridine diphosphate glucose (UDP-glucose) or guanosine diphosphate mannose (GDP-mannose). Because glycosylation influences properties and mechanisms of action of pharmaceutically relevant natural products including cell recognition (Weymouth-Wilson, 1997, Nat Prod Rep. 14(2):99-110., Thorson et al., 2001, Curr Org Chem 5, 139-167), the synthesis of oligosaccharides and glycosylated products continues to promise a vast variety of new carbohydrate structures with potential applications in medicine and industry. However, as noted by Lim et al. (Biotechnol. Bioeng.. 87: 623-631 (2004)), “a major constraint on the use of glycosyltransferases in small molecular biocatalysis has been the apparent need for UDP-glucose.” Because of the difficulty in enzymatic recycling of glyconucleotides, whole cell conversions appear necessary for the large scale donation of any sugar residue to various organic molecules. The demand for glyconucleotides is particularly pressing for the production of gram-quantities of glucosides. The glucose accumulation as demonstrated in our preliminary research can readily be directed to the formation of UDP-glucose and potentially other glyconucleotides for the production of a vast array of glucosides. As an example of one reaction mediated by GTs using UDP-glucose, FIG. 6 illustrates the formation of the anthocyanin cyanidin 3-O-glucoside from the anthocyanidin cyanidin.

Glycosylated products via fermentation have been the focus of much previous research. For example, Yan et al. (Appl Environ Microbiol 71, 3617-3623 (2005)) generated cyanidin 3-O-glucoside and pelargonidin 3-O-glucoside using a four step pathway from the flavanone precursors naringen and eriodictyol supplied at 1 mM. For these studies, 5 mM UDP-glucose was added to the medium, and the final concentrations of the target anthocyanins achieved after 65 h were at most 6 μg/L (13 nanomolar). Thus, UDP-glucose was added to the medium in 106-fold excess of the final product glyconucleotide concentration. Other studies have attempted also to generate these aglycon precursors using P450 monooxygenases whose functional expression is challenging (Hotze et al., 1995, FEBS Lett 374, 345-350), or enzymes which altogether bypass cytochrome P450 hydroxylase (Hwang et al., 2003, Appl Environ Microbiol 69, 2699-2706). Another approach has been to express glyconucleotide synthase genes in strain lacking the pgi gene (FIG. 1 [D]), which makes glucose-6P more available than a wild-type strain (Simkhada et al., 2010, Biotechnol Bioeng 107, 154-162; Kurumbang et al., 2010, J Appl Microbiol 108, 1780-1788). However, formation of glyconucleotides and subsequent use of glycotransferases has not been accomplished in a strain which actually accumulates gram-quantities glucose. A process to direct (accumulated) glucose to UDP-glucose or other glyconucleotides serves as a platform to benefit broadly the formation of all glycosylated products by any of these means. See also DeBruyn et al., Biotechnol. Bioeng., 112(8): 1594-1603 (2015) for a description of glucosyl transferases, glycosylation and glucoside products that can be produced using the present invention.

GTs are often promiscuous with respect to their acceptor. For example, the mannosylglycerate synthase enzyme from Rhodothermus marinus can transfer a mannose from GDP-mannose to various small β-hydroxy acids such as glycerate, lactate and glycolate (Flint et al., 2005). GTs have been evolved to extend the type of glycosylation and increase their range of donors, such as the widely studied mutant oleD GT from Streptomyces antibioticus which glycosylates phenol (O-glycosylation), thiophenol (S-glycosylation) and aniline (N-glycosylation) and also catalyzes iterative glycosylation (Gantt et al., 2008. Ange Chem Intl Ed 47, 8889-8892).

Glycosylated compounds are not the only products which could be formed in a system which accumulates hexoses. Another class of compounds derived from sugars is the rare sugars (Beerens et al., 2012, J Ind Microbiol Biotechnol 39, 823-834). Only seven sugars actually exist in sufficient amounts to be extracted directly from natural sources (D-glucose, D-galactose, D-mannose, D-fructose, D-xylose, D-ribose, and L-arabinose). A larger group of hexoses, pentoses and deoxygenated monosaccharides (most L-isomers, and others such as D-idose, D-psicose and D-lyxose) exist in small quantities in nature but play a crucial role in bioactive recognition elements. Often these sugars impart increased antiviral activity, better metabolic stability and more favorable toxicological profiles than common sugars. For example, L-talose nucleotides are inhibitors against leukemeia cells (Lerner and Mennitt, 1994), L-ribose has potential against HBV and Epstein-Barr virus (Tianwei et al., 1996, J Med Chem 39, 2835-2843), L-lyxose is a component of the antibiotic avilamycin A (Hofmann et al., 2005, Chem Biol 12, 1137-1143), and L-gulose is used in the synthesis of potent HBV and HIV inhibitors (Dondoni et al., 1997, J Org Chem 62, 6261-6267). These sugars have to be produced synthetically or enzymatically using epimerases, oxidoreductases and isomerases. The in vitro enzymatic or chemical synthesis of these sugars is costly. Advantageously, the intracellular accumulation of hexoses such as fructose and glucose provides a means to generate several of these compounds using microbial processes with a potential for substantial decrease in cost.

Advantageously, the instant invention can be extended to the accumulation of other products which can be derived from hexoses, but which were either not considered previously or were not feasible because the substrate (i.e., the hexose) scarcely accumulated intracellularly at sufficient concentrations to drive the necessary enzymatic processes. Examples of compounds that can be derived from hexoses include, without limitation, a wide variety of glycosylated (glucuro-, gluco-, galacto-, manno-derivatized) compounds, and dihexose sugars (formed with a condensation reaction to form a 1,6-glycosidic bond).

Incorporation of Cl compounds

In still other aspects, this invention can be extended to a means of building onto a carbon backbone. For example, a hexose-derived product can be formed via the incorporation of Cl compounds into hexoses such as glucose. The formation and accumulation of hexoses in aerobic microbial cells may lead to processes analogous to those in plants for the sequestration of 1-carbon compounds, with the inherent advantages of high volumetric rates and the absence of light or anoxic requirements. Hexose accumulation may be an important means of storing energy from lower molecular weight carbon compounds.

Substantial academic and industrial interest exists in incorporating Cl-compounds (e.g., CO₂, CO, formate, formaldehyde, methane and methanol) into central metabolism. Several Clostridium spp. use the Wood-Ljungdahl pathway to assimilate CO and CO₂ (Bruant et al., 2010, PLoS one 5, 9). Moorella thermoacetica (Clostridium thermoaceticum) uses formate dehydrogenase to convert CO₂ to formate (using NADPH). Other organisms use metabolic cycles to incorporate Cl compounds including the xylulose-5P (Xu5P) cycle, the ribulose-5P (Ru5P) cycle, the serine cycle, and the ribulose-1,5P2 pathway (i.e., the Calvin cycle), as well as less-studied 3-hydroxypropionate/malyl CoA cycle and the 4-hydroxybutyrate cycle (Herter et al., 2001, J Bacteriol 183, 4305-4316; Jahn et al., 2007, J Bacteriol 189, 4108-4119).

The relatively insoluble gases CO and methane, and to a lesser extent the more soluble CO₂, have the inherent problem of mass transport of the gas into the liquid phase to be metabolized by cells, a challenge which generally necessitates a continuous oversupply of a gas in order to provide an adequate driving force. Formaldehyde and methanol are enzymatically interconvertable but the latter is preferred as the microbial feedstock hexose formation from supplemented Cl-compounds because formaldehyde is comparatively toxic to cells. Methanol can be incorporated into glucose via its co-metabolism with xylose and arabinose. One should note, however, that methanol is not the only suitable Cl feedstock. Chemical processes for the oxidation of methane and the reduction of CO₂ are being vigorously investigated by others. For example, selective oxidation of light alkanes to oxygenates with oxygen has been carried out over numerous catalysts--oxidation of methane to formaldehyde using carbon dioxide as the oxidant has been demonstrated over a V₂O₅ catalyst (Shimamura et al., 2004, J Molecul Catal A 211, 97-102):

CH₄+2 CO₂→HCHO+2CO+H₂O

Also, Matsumura et al. (2006, J Molec Catal A 250, 122-130) were able to demonstrate the selective oxidation of methane to formaldehyde on a Sb2O4 catalyst:

CH₄+O₂→HCHO+H₂O

Ruthenium complexes are efficient catalysts for the conversion of H₂/CO₂ mixtures into formate (Koike and Ikariya, 2004; Ohnishi et al., 2005, . J Amer Chem Soc 127, 2021-4032), and methanol can be generated by the reduction of CO₂ with borane (Chakraborty et al., 2010, J Amer Chem Soc 132, 8872-8873).

Methanol can be incorporated into central metabolism by first oxidizing the alcohol to formaldehyde. Methylococcus capsulatus and other methanotrophs metabolize formaldehyde by the cyclic Ru5P pathway in which as the first steps formaldehyde is condensed with a pentose phosphate to form a hexose phosphate:

3 formaldehyde+3 D-ribulose-5P→3 D-fructose-6P [5]

3-Hexulose-6P synthase (EC 4.1.2.43, HPS) and 6P-3-hexuloisomerase (EC 5.3.1.27, PHI) are the two characteristic enzymes involved in the Ru5P cycle for formaldehyde (or methanol) fixation. HPS catalyzes the conversion of formaldehyde and D-ribulose-5P to D-arabino-hex-3-ulose 6-phosphate (hexulose-6P), and PHI catalyzes the isomerization between hexulose-6P and D-fructose-6P. The genes for several HPS and PHI enzymes have been sequenced and cloned. For example, the hps gene encoding HPS has been cloned from the obligate methylotroph Methylomonas aminofaciens (Yanase et al., 1996, FEMS Microbiol Lett 135, 201-205), an enzyme which has a very low KM of 0.29 mM, below the ˜1-2 mM tolerance of microorganisms for formaldehyde (Kato et al., 1978, Biochim Biophys Acta 523, 236-244). The enzyme from a Bacillus spp. has a KM value of 0.15 mM (Kato et al., 1978, Biochim Biophys Acta 523, 236-244). The rmpB gene encoding PHI has been cloned from M. aminofaciens (Sakai et al., 1999, FEMS Microbiol Lett 176, 125-130) and the facultative methylotroph Mycobacterium gastri (Mitsui et al., 2000. J. Bacteriol 182, 944-948). The hxlA and hxlB genes encoding respectively HPS and PHI from the nonmethylotroph Bacillus subtilis have also been expressed in E. coli and characterized (Yasueda et al., 1999, J. Bacteriol 181, 7154-7160). The formation of a six-carbon sugar phosphate instead of two 3-carbon sugar phosphates distinguishes the Ru5P cycle from the Xu5P cycle, and makes the former cycle ideal for accumulating hexoses like glucose. In still other embodiments, the instant invention can be extended to the accumulation of specific building-block compounds derived from intermediates of the PP pathway (e.g., phenylalanine, histidine, ribose).

### Other Embodiments

In one aspect, the invention provides for microbial conversion of a pentose (e.g., xylose or arabinose) to a hexose (e.g., glucose). It should be understood, however, that other sugar interconversions are also contemplated. The basic strategy for hexose formation relies on knocking out a specific sugar uptake system and then diverting carbon to that unmetabolizable sugar. For example, the conversion of fructose to glucose can be achieved by eliminating glucose consumption (as already shown), and then curtailing fructose utilization, for example by knocking out one or more of several genes: pfkA and zwf as above, mak (mannokinase, FIG. 1[K]),fruK (fructose-1P kinase , FIG. 1[M]),fbaA/bfaB (fructosbisphophate aldolase, FIG. 1[P]), and intriguingly talA/talB (transaldolase, FIG. 1[I]). Some of these knockouts may, optionally, benefit from supplementing the medium with a secondary carbon source such as glycerol or acetate, which can be used as growth-limiting substrates as described above. Similarly, for the conversion of glucose to fructose, we would eliminate the fructose PTS system (levF levG fruA), and direct more glucose to fructose: knockouts in zwf and pfkA as above, as well as talA/talB; and other genes such as xylA or agp, depending on results above.

Exemplary biological sources of genes and enzymatic activities are described herein. For example, Escherichia coli and its pentose pathway were used to construct the strains that exemplify the invention. However, it should be understood that what is important is that the microbial cell is engineered to produce a hexose as a final product; the actual biological sources are not important and can be determined by the skilled artisan based on availability or convenience.

In the preceding description, particular embodiments may be described in isolation for clarity. For example, exemplary embodiments provided herein focus on the conversion of xylose to glucose in Escherichia coli. However, it should be understood that the invention includes the conversion of any pentose or glycerol to any hexose and may be accomplished using any suitable microbial cell. Unless it is otherwise expressly specified that the features of one particular embodiment are incompatible with the features of another embodiment, the invention is intended to encompass embodiments which include a combination of two or more compatible features described herein in connection, regardless of the textual position of the description of those embodiments within the document.

Moreover, it should be understood that preceding description is not intended to disclose every embodiment or every implementation of the present invention. The description more particularly exemplifies illustrative embodiments.

In several places throughout the application, guidance is provided through lists of examples, which examples can be used in various combinations. In each instance, the recited list serves only as a representative group and should not be interpreted as an exclusive list.

For any method disclosed herein that includes discrete steps, the steps may be conducted in any feasible order. And, as appropriate, any combination of two or more steps may be conducted simultaneously.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

### Accumulation of D-Glucose from Pentoses by Escherichia coli

### Abstract

Escherichia coli unable to metabolize D-glucose (knockouts in ptsG, manZ, glk) accumulates a small amount of D-glucose (yield of about 0.01 g/g) during growth on the pentoses D-xylose or L-arabinose as a sole carbon source. Additional knockouts in zwf and pfkA genes encoding respectively D-glucose-6-phosphate 1-dehydrogenase and 6-phosphofructokinase I (E. coli MEC143) increased accumulation to greater than 1 g/L D-glucose and about 100 mg/L D-mannose from 5 g/L D-xylose or L-arabinose. Knockouts of other genes associated with interconversions of D-glucose-phosphates demonstrate that D-glucose is formed primarily by the dephosphorylation of D-glucose-6P. Under controlled batch conditions with 20 g/L D-xylose, MEC143 generated 4.4 g/L D-glucose and 0.6 g/L D-mannose. The results establish a direct link between pentoses and hexoses, and provide a novel strategy to increase carbon backbone length from five to six carbons by directing flux through the pentose phosphate pathway. See, e.g., Xia et al., 2015, Appl. Environ. Microbiol. 81:3387-3394.

### Introduction

The pentose phosphate (PP) pathway interconverts phospho-sugars having 3-7 carbon atoms principally by the action of the reversible enzymes transketolase and transaldolase. During the consumption of hexoses such as D-glucose or D-fructose, entry of carbon into this pathway provides many microorganisms including Escherichia coli the means to generate the reduced co-factor NADPH and to synthesize specific building-block compounds derived from intermediates of this pathway (e.g., phenylalanine, histidine, ribose). For microorganisms having the requisite kinases and sugar transport mechanisms, the PP pathway also provides convenient entry points for the catabolism of many other sugars including D-xylose and L-arabinose.

We have previously studied D-xylose and L-arabinose metabolism in E. coli that lacks the ability to metabolize D-glucose due to knockouts in the ptsG, manZ and glk genes (15, 16, 48). Recently, small but consistent amounts (about 50 mg/L) of D-glucose were observed as the accumulated end-product when E. coli ptsG manZ glk was grown on 5 g/L of either pentose in a defined medium (unpublished). How might D-glucose be derived from these pentoses?

Both D-xylose and L-arabinose are converted to the common intermediate D-xylulose-5P (FIG. 2), which via the PP pathway partitions to 67% D-fructose-6P and 33% D-glyceraldehyde-3P without the involvement of ATP:

3 D-xylulose-5P→2 D-fructose-6P+D-glyceraldehyde-3P [2]

During growth of cells having a complete glycolytic pathway, the 2 moles of D-fructose-6P formed via Eq. 2 readily generates 4 moles of D-glyceraldehyde-3P. For D-glucose to accumulate from pentoses in cells prevented from metabolizing D-glucose, we reasoned that some D-fructose-6P generated from these pentoses (i.e., by Eq. 2) is converted “back” to D-glucose, and that once formed, the D-glucose was unable to reenter metabolism in the triple knockout strain. We furthermore hypothesized that even more D-glucose would accumulate from pentoses in cells that were further constrained from metabolizing D-fructose-6P or D-glucose-6P.

Because D-fructose-6P conversion to D-glyceraldehyde-3P is ubiquitous in wild-type organisms, D-glucose is not typically considered a product of D-xylose or L-arabinose metabolism, and the conversion of these pentoses to readily available D-glucose would in itself not seem to be an economically viable process. However, if the yields and rates were sufficiently large, the accumulation of hexoses directly from pentoses might advance the use of lignocellulosic hydrolysates with organisms such as Saccharomyces cerevisiae which metabolize D-glucose readily but are natively unable to consume pentoses. Moreover, conversion of 5-carbon saccharides into 6-carbon saccharides derived from D-fructose-6P offers a unique platform both to build carbon length and potentially to generate compounds in industrially relevant organisms such as E. coli that might not be possible under typical conditions in which products of D-fructose-6P do not accumulate.

The objectives of this study were to examine D-glucose formation from the pentoses D-xylose and L-arabinose. Specifically, we sought to identify the pathway involved in the formation of D-glucose from pentoses and to increase further the formation of D-glucose by preventing D-fructose-6P and D-glucose-6P metabolism. Finally, under the controlled conditions of a bioreactor we examined if elevated concentrations of D-glucose could be synthesized from either D-xylose or L-arabinose as sole carbon sources.

### Materials and Methods

### Bacterial Strains

Escherichia coli ALS1048 (MG1655 ΔptsG763::(FRT) ΔmanZ743::(FRT) Δglk-726::(FRT)) was used to construct additional strains as listed in Table 1 (16). These strains were constructed by transducing ALS1048 with the corresponding Keio (FRT)Kan deletions (2), and if necessary, curing the Kan(R) using the pCP20 plasmid, which contains a temperature-inducible FLP recombinase as well as a temperature-sensitive replicon (12). All strains were verified by PCR.

TABLE 1
E. coli strains used in this study.

| Strain | Genotype | Reference |
|---|---|---|
| ALS1048 | MG1655 ΔptsG763::(FRT) | Eiteman et al., 2009 |
| | ΔmanZ743::(FRT) Δglk-726::(FRT) | |
| MEC132 | ALS1048 ΔpfkA775::Kan | This study |
| MEC143 | ALS1048 ΔpfkA775::(FRT) | This study |
| | Δzwf-777::Kan | |
| MEC144 | ALS1048 Δzwf-777::Kan | This study |
| MEC151 | ALS1048 ΔpfkA775::(FRT) | This study |
| | Δzwf-777::(FRT) Δmak-759::Kan | |
| MEC152 | ALS1048 ΔpfkA775::(FRT) | This study |
| | Δzwf-777::(FRT) Δagp-746::Kan | |
| MEC178 | ALS1048 ΔpfkA775::(FRT) | This study |
| | Δzwf-777::(FRT) Δgcd-742::Kan | |
| MEC180 | ALS1048 ΔpfkA775::(FRT) | This study |
| | Δzwf-777::(FRT) ΔxylA748::Kan | |
| MEC319 | ALS1048 ΔpfkA775::(FRT) | This study |
| | Δzwf-777::(FRT) Δpgm-736::Kan | |
| MEC320 | ALS1048 ΔpfkA775::(FRT) | This study |
| | Δpgi-721::Kan | |
| MEC321 | ALS1048 ΔpfkA775::(FRT) | This study |
| | Δzwf-777::(FRT) Δpgi-721::Kan | |

In one experiment the pgi gene encoding E. coli phosphoglucose isomerase was overexpressed. To construct the pTrc99A-pgi plasmid, the pgi gene was PCR amplified with primers 5′-GGGAAAGAATTCAAAAACATCAATCCAACGCAGACCGC-3′ (SEQ ID NO:1) (forward) and 5′-GGGAAAGGATCCTTAACCGCGCCACGCTTTATAGCG-3′ (SEQ ID NO:2) (reverse) using E. coli BW25113 genomic DNA as the template. The 1,671 by PCR product was purified, restricted with EcoRI and BamHI and ligated into the regulable expression vector pTrc99A that had also been restricted with EcoRI and BamHI to yield the plasmid pTrc99A-pgi, which was subsequently transformed into MEC320 and MEC321.

### Growth Medium and Conditions

The defined medium used for the shake flask experiments contained (per liter): 1.70 g citric acid, 13.30 g KH₂PO₄, 4.50 g (NH₄)₂HPO₄, 1.2 g MgSO₄·7H₂O, 13 mg Zn(CH₃COO)₂·2H₂O, 1.5 mg CuCl₂·2H₂O, 15 mg MnCl₂·4H₂O, 2.5 mg CoCl₂·6H₂O, 3.0 mg H₃BO₃, 2.5 mg Na₂MoO₄·2H₂O, 100 mg Fe(III) citrate, 4.5 mg thiamine·HCl, 8.4 mg Na₂(EDTA)·2H₂O, and 5.0 g D-xylose, L-arabinose, glycerol or D-fructose. The pH was adjusted to 7.0 with 30% (w/v) NaOH. Cells were routinely stored on Lysogeny Broth (LB) agar plates, transferred to 1 mL of LB medium in a test tube overnight, then 20 mL defined medium in a 250 mL shake flask, from which 2 mL was transferred to the 50 mL defined medium in a 250 mL shake flask used for these studies. Shake flask studies were replicated 3-6 times for each strain and pentose when D-glucose was detected. Statistical analyses were completed using Student's t-test (two-tailed, equal variance), and p<0.10 was considered the criterion for significance. For larger scale studies in a controlled bioreactor, the sequence of transfers was identical, and the 50 mL from the final shake flask was used to inoculate the larger vessel. The flasks were incubated at 37° C. with an agitation of 250 RPM. Samples were stored at −20° C. for subsequent analysis.

A single controlled batch process at 1.0 L volume was carried out using D-xylose in a 2.5 L bioreactor (Bioflo 2000, New Brunswick Scientific Co. Edison, N.J., USA). The same defined medium was used except the concentration of D-xylose was 20 g/L. Air or oxygen as necessary was sparged into the fermenter with the agitation set at 500 rpm to maintain the dissolved oxygen above 40% saturation. The pH was controlled at 7.0 using 20% NaOH, and the temperature was controlled at 37° C. Antifoam C (Sigma) was used as necessary to control foaming.

Continuous processes using MEC143 operated as nitrogen(N)-limited chemostats at 1.0 L volume were conducted in the same 2.5 L fermenter. To ensure N-limitation and prevent contamination, the medium contained (per L) 1.0 g (NH₄)₂HPO₄ (15 mM N), 8.0 g D-xylose and 40 mg kanamycin, but otherwise remained unchanged. Four dilution rates (growth rates) were examined in the range 0.08-0.15 ⁻¹, and a steady-state condition was assumed after four residence times at which time the oxygen and CO₂ concentrations in the effluent gas appeared constant. These processes were conducted at 37° C. with an air flowrate of 0.5 L/min, an agitation of 400 rpm and a pH of 7.0. The dissolved oxygen remained above 40% saturation. A carbon balance was completed using a unit carbon formula weight for E. coli cell mass of 24.6 g/mol (5).

### Analytical Methods

The optical density at 600 nm (OD) (UV-650 spectrophotometer, Beckman Instruments, San Jose, Calif.) was used to monitor cell growth. Liquid chromatography with a refractive index detector and a Coregel 64-H ion-exclusion column (Transgenomic Ltd., Glasgow, United Kingdom) using a mobile phase of 4 mN H₂SO₄ was used for analysis of sugars and acetic acid as described previously (14). For dry cell weight measurement, three 25.0 mL samples were centrifuged (8400×g, 10 min), the pellets washed by vortex mixing with 30 mL 0.9% saline solution and then centrifuged again. After repeating the washing step twice using DI water, the cell pellets were dried at 60° C. for 24 h and weighed. The concentrations of oxygen and CO₂ in the off-gas were measured using a gas analyzer (Innova 1313 gas monitor, Lumasense Technologies, Ballerup, Denmark).

The presence of sugars was confirmed by comparing samples with standards using a derivatization protocol with a GC-MS (6). The GC-MS method was used only for identification and not quantification, in particular those cases in which the analytes eluted closely by HPLC (D-mannose and D-xylose) or to confirm the absence of a sugar (e.g., D-fructose). Briefly, samples were centrifuged, the supernatant evaporated to dryness, and then derivatized with 700 μL hexamethyldisilazane, 200 μL anhydrous pyridine, and 10 μL trifluoroacetic acid at 60° C. for 3 h. Detection of derivatized analytes was accomplished with a GC-MS (HP6890/HP5973, electron ionization energy of 70 eV, Agilent Technologies, Inc., Santa Clara, Calif. USA). One microliter (1 μL) was injected onto a 30 m HP-5MS column (Agilent Technologies, Inc.) in the split flow mode, 30:1 with 1 mL/min flow rate. The temperature profile began at 50° C. for 1 min, increased at 2° C./min to 100° C., increased at 5° C./min to 250° C., and held for 5 min. Injector temperature was 250° C., MS source was 230° C., MS Quad was 150° C., and the GC-MS interface was 280° C. For the N-limited chemostats, the ammonia nitrogen (NH₄-N) in feed and effluent was analyzed using the colorimetric EPA Method 350.1 (44).

We used NMR to demonstrate that D-glucose was formed biologically and accumulated in the medium. Four samples were analyzed: a glucose standard, a D-glucose-6P standard, a sample from a shake flask experiment (i.e., containing D-glucose), and a D-glucose-6P standard incubated in sterile medium for 24 h at 37° C. NMR data were acquired using a Varian INOVA instrument with a cryogenic probe system at 14.1 T (600 MHz ¹H). The sample temperature was maintained at 25° C. Standard, natural abundance, two-dimensional ¹H, ¹³C-HSQC spectra were acquired in the constant-time (¹³C decoupled) mode. Chemical shift assignments were made by reference to database entries and published works (3, 9, 38, 39, 40, 43). The ¹H chemical shifts were referenced with respect to external Na⁺DSS⁻ (sodium 4,4-dimethyl-4-silapentane-1-sulfonate) in D₂O at 25° C. (0.0 ppm). The ¹³C chemical shifts were referenced indirectly assuming the absolute frequency ratio: ¹³C/¹H=0.251449530 (47). D₂O was added to samples to a final concentration of approximately 7% for instrumental lock. NMR data were processed and signal intensities measured using Felix (Accelrys, San Diego, Calif.).

### Results

Formation of Glucose from Xylose or Arabinose

E. coli ALS 1048 contains knockouts in the ptsG, manZ and glk genes and is unable to metabolize D-glucose (16). Using this strain as a baseline for comparison, we first sought to determine whether additional D-glucose would accumulate from either D-xylose or L-arabinose if D-fructose-6P and D-glucose-6P were prevented from entering glycolysis and the PP pathway. Specifically, we first constructed strains with additional knockouts in pfkA encoding 6P-fructokinase I (EC 2.7.1.11) and/or zwf encoding D-glucose-6P 1-dehydrogenase (EC 1.1.1.49). During growth in shake flasks using either 5 g/L D-xylose or 5 g/L L-arabinose, ALS1048 and ALS1048 zwf(MEC144) accumulated D-glucose at a yield of about 0.01 g/g from either pentose (FIG. 3), while ALS1048 pfkA (MEC132) generated D-glucose at yields of 0.13 g/g from D-xylose and 0.17 g/g from L-arabinose. Eliminating both pathways in ALS 1048 pfkA zwf (MEC143) resulted in the accumulation of D-glucose at yields of 0.26-0.29 g/g. For both pfkA-knockout strains MEC132 and MEC143, we also observed the formation of 60-180 mg/L D-mannose using HPLC and confirmed by GC-MS (see Materials and Methods). No other product such as D-fructose was identified by GC-MS. Moreover, the D-glucose and D-mannose were not metabolized within several hours after the pentose was exhausted. These results clearly show that E. coli can generate D-glucose from pentoses through D-fructose-6P, suggesting a route for the formation of 6-carbon products from 5-carbon substrates by preventing the intermediate D-fructose-6P from entering glycolysis and the PP pathway (FIG. 2). We also repeated the identical shake flask experiments using MEC143 with 5 g/L glycerol or 5 g/L D-fructose, and observed D-glucose as a final product at a concentration of about 60 mg/L or 75 mg/L, respectively (yield of about 0.01 g/g).

### Identification of Key Enzymes Involved in Glucose Formation

By preventing D-glucose utilization in E. coli while simultaneously blocking entry of D-fructose-6P into glycolysis and re-entry into the PP pathway, significant D-glucose formed from D-xylose or L-arabinose (FIG. 3). We therefore sought next to clarify the pathway E. coli uses to convert D-fructose-6P to D-glucose by constructing additional knockout strains.

The formation of some D-mannose during the accumulation of D-glucose suggests the involvement of D-mannose as a pathway intermediate. Also, one possible route for D-glucose formation from D-fructose-6P would be via D-fructose. The enzyme mannokinase (EC 2.7.1.4) encoded by mak is known to phosphorylate D-mannose and D-fructose (42). We therefore hypothesized that mannokinase might be involved in the accumulation of D-glucose from pentoses via the conversion of D-fructose-6P to D-fructose. However, E. coli ALS 1048 pfkA zwf mak (MEC151) did not show any difference in D-glucose formation from either pentose compared to MEC143 (FIG. 3). Also, D-mannose was formed as before (230-270 mg/L), supporting the conclusion that mannokinase is not involved in the formation of either D-mannose or D-glucose from pentoses.

Another pathway that potentially could serve to form D-glucose is via the enzyme xylose isomerase (EC 5.3.1.5). In addition to interconverting D-xylose and D-xylulose, the E. coli xylose isomerase interconverts D-fructose and D-glucose (7, 17, 46), but less efficiently (41). Though the K_{M} values for D-fructose, D-glucose and D-xylose have not been reported for the E. coli enzyme, the k_{CAT} values for D-glucose and D-fructose are similar for the enzyme from other organisms (29), suggesting that D-fructose and D-glucose both readily serve as substrates for this isomerization. We therefore knocked out the xylA gene to form strain ALS 1048 pfkA zwf xylA (MEC180). Of course, the xylA knockout also renders this strain unable to consume D-xylose, and therefore only the conversion of L-arabinose to D-glucose could be examined. The deletion of xylose isomerase reduced D-glucose yield only slightly to 0.23 g/g (FIG. 3), and D-mannose was detected at 170-190 mg/L, corresponding to a yield of about 0.03 g/g. These results suggest xylose isomerase does not play a significant role in the formation of both D-glucose and D-mannose from pentoses.

We next examined one possible route through which D-glucose could be utilized. The gcd gene encoding glucose dehydrogenase (EC 1.1.5.2) is able to convert D-glucose into D-glucono-1,5-lactone which can then spontaneously form gluconate (45), although pyrroloquinoline quinone appears to be necessary for this conversion in E. coli (31). In order to determine whether D-glucose accumulation is influenced by glucose dehydrogenase, we constructed ALS1048 pfkA zwf gcd (MEC178). MEC178 formed D-glucose from D-xylose (yield of 0.28 g/g) or from L-arabinose (0.30 g/g), and also formed about 110-150 mg/L D-mannose from either pentose (0.03 g/g), indicating that this route does not significantly affect hexose formation (FIG. 3).

We next examined whether the formation of D-glucose was the result of the hydrolysis of either D-glucose-1P or D-glucose-6P. D-Fructose-6P is converted to D-glucose-6P by glucose-P isomerase encoded by pgi, D-glucose-6P is converted to D-glucose-1P by phosphoglucomutase encoded by pgm, and D-glucose-1P can be dephosphorylated by D-glucose 1-phosphatase encoded by agp (FIG. 2). The D-glucose yield was unchanged as a result of the agp knockout (ALS 1048 pfkA zwf agp, MEC152), and was 0.18-0.20 g/g with a pgm knockout (ALS 1048 pfkA zwfpgm, MEC319). Both MEC152 and MEC319 accumulated 130-170 mg/L D-mannose from 5 g/L of either pentose. However, D-glucose and D-mannose formation were completely eliminated as a result of the pgi knockout (ALS 1048 pfkA zwfpgi, MEC321, FIG. 3). Because MEC319 showed a slight reduction in D-glucose yield, the results do not exclude the possibility of some D-glucose-1P hydrolysis resulting in D-glucose formation, although the lower observed glucose yield in MEC319 compared to MEC143 could also be simply due to the cells' reduced ability to form necessary metabolites from D-glucose-1P and UDP-D-glucose. The complete elimination of D-glucose formation as a result of a pgi deletion supports the conclusion that the hydrolysis of D-glucose-6P is the principal final step by which D-glucose is formed from pentoses.

Two additional experiments were conducted to confirm the role of pgi in D-glucose formation. First, because the pgi knockout blocks D-glucose-6P formation (FIG. 2), the zwf knockout should not affect D-glucose formation in a pgi knockout. In other words, the ptsG manZ glk pfkA pgi strain should also be unable to accumulate D-glucose. We therefore examined ALS 1048 pfkA pgi (MEC320), and indeed observed no D-glucose formation from either D-xylose or L-arabinose (FIG. 3). Second, we transformed both MEC320 and MEC321 with pTrc99A-pgi expressing native phosphoglucose isomerase, and these strains regained the ability to accumulate D-glucose from either D-xylose or L-arabinose. MEC320 pTrc99A-pgi attained a yield of 0.09 g/g and MEC321 pTrc99A-pgi attained a yield of 0.13 g/g. Interestingly, knockout strains which generated more than 0.05 g/g D-glucose accumulated significantly more D-glucose from L-arabinose than from D-xylose (p<0.10, FIG. 3) except MEC178 (ALS1048 pfkA zwf gcd) for which there was no significant difference. For example, MEC143 (ALS1048 pfkA zwf) generated 27% more D-glucose from L-arabinose than from D-xylose.

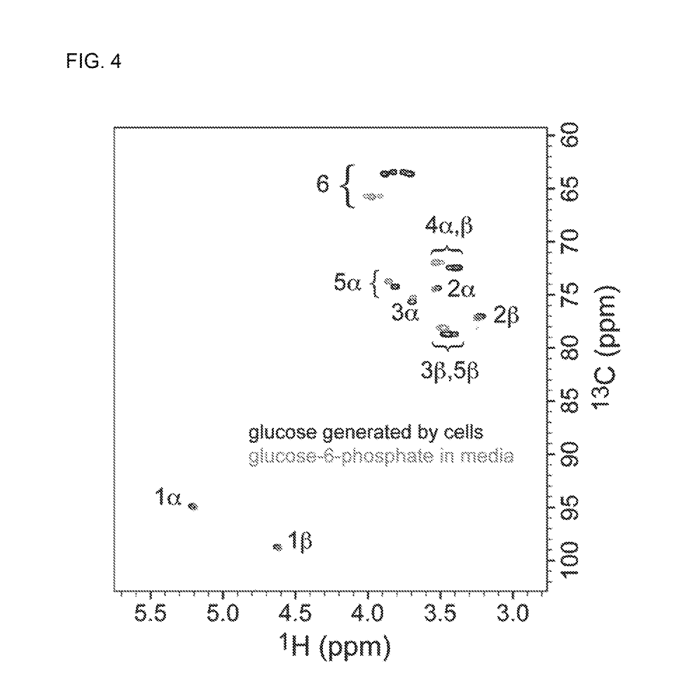

Finally, we confirmed D-glucose was the biological product from both pentoses by comparing the NMR spectra of D-glucose and D-glucose-6P, and also by demonstrating that D-glucose could not have formed from D-glucose-6P by chemical hydrolysis within the medium nor during the HPLC method at the temperatures used (FIG. 4). The NMR results confirm that extracellular D-glucose and not D-glucose-6P was the biological product of D-xylose or L-arabinose metabolism in these knockout strains.

### Batch Process to Accumulate Glucose

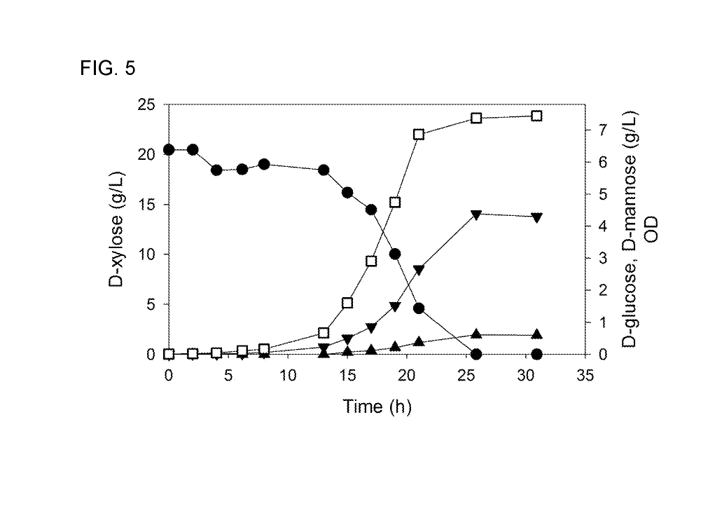

The previous experiments were all conducted in shake flasks using 5 g/L L-arabinose or D-xylose. We next conducted an experiment using MEC143 in a controlled bioreactor with approximately 20 g/L D-xylose, to determine if a proportionate increase in D-glucose (and D-mannose) accumulation would be observed. In this batch run, about 4.4 g/L D-glucose and 0.61 g/L D-mannose were formed in 25 h for an observed mass yield from D-xylose of 0.21 g D-glucose/g and 0.03 g D-mannose/g (FIG. 5). Furthermore, neither D-glucose nor D-mannose was reassimilated 5 h after D-xylose was exhausted. A nearly proportionate increase in product formation was observed in these controlled processes compared to the shake flask studies, suggesting that D-glucose formation is not inhibited nor repressed by D-glucose accumulation.

This result also demonstrates a potential for generating substantial quantities of 6-carbon hexoses from 5-carbon pentoses.

### Continuous Processes to Accumulate Glucose

Chemostats are a convenient tool to study microbial growth and product formation under nutrient-limited conditions. During the batch process previously studied the cells were grown under nutrient-excess conditions, and we reasoned that carbon flux might be maximal if the cells were grown under conditions for which growth was limited by a nutrient other than carbon. Furthermore, being at steady-state and at a controlled growth rate, a chemostat would demonstrate whether the D-glucose observed is formed as a transient product or only during maximal cell growth. We therefore next grew MEC143 under nitrogen-limiting conditions by increasing the concentration of D-xylose and decreasing the concentration of the nitrogen source (see Materials and Methods). At four different dilution rates (D=0.08 h⁻¹ to 0.15 h⁻¹), the observed yields averaged 0.26 (±0.08, standard deviation) g D-glucose/g D-xylose and 0.23 (±0.03) g dry cells/g D-xylose, and these values did not vary with dilution rate. The mean carbon recovery was 108% (±11%), 2.3-3.3 g/L D-xylose and less than 0.5 mg/L N were detected in the effluents. These results demonstrate that D-glucose formation is not a transient phenomenon. Since the yields during the nitrogen-limited chemostats were similar to yields observed in batch processes, D-glucose does appear to form as an overflow metabolite.

### Analysis

During growth on D-xylose or L-arabinose, wild-type E. coli generates 2 moles of D-fructose-6P and 1 mole D-glyceraldehyde-3P from 3 moles of either pentose (Eq. 2). If the glycolytic pathway is complete, the 2 moles of D-fructose-6P formed via Eq. 2 readily generates 4 moles of D-glyceraldehyde-3P. Indeed, because the conversion of D-fructose-6P to D-glyceraldehyde-3P is readily accomplished in widely-studied microorganisms, D-fructose-6P is typically not thought of as an intermediate of pentose metabolism. However, our results demonstrate that E. coli can direct this metabolic intermediate D-fructose-6P into other 6-carbon (i.e., hexose) end-products such as D-glucose when three conditions are met.

A first condition for the accumulation of products derived from pentoses via D-fructose-6P is that glycolysis must be disrupted between D-fructose-6P and D-glyceraldehyde-3P. By blocking glycolysis, the pentose phosphate pathway essentially becomes a branched pathway during the metabolism of D-xylose or L-arabinose with two separate products, D-fructose-6P and D-glyceraldehyde-3P (FIG. 2). That is, when D-fructose-6P cannot enter glycolysis it becomes available for the formation of other 6-carbon products, while the D-glyceraldehyde-3P remains available for the generation of ATP, NADH and the precursors that exist metabolically “below” D-glyceraldehyde-3P via the terminal steps of glycolysis and the tricarboxylic acid cycle. In E. coli the entry of D-fructose-6P into glycolysis can be blocked by a deletion in the pfkA gene.

A second condition to facilitate the accumulation of hexoses from pentoses is that metabolites should be prevented from re-entering the PP pathway, for example, from D-glucose-6P. In E. coli the re-entry of D-glucose-6P into the PP pathway can be prevented by a knockout of the zwf gene (FIG. 2). Finally, as a third condition the ultimate product must be excreted and should not be re-metabolized. Our results demonstrate that the knockouts in the ptsG, manZ and glk genes effectively block D-glucose metabolism and at least curtail its reassimilation once generated.

E. coli MEC143, which met these three conditions, accumulated significant D-glucose from either of two pentoses, L-arabinose or D-xylose. Interestingly, D-glucose was also observed, but to a much lesser extent, when glycerol or D-fructose was the sole carbon source in the same strain, probably as a result of the formation of a small quantity of D-fructose-6P generated via the non-oxidative PP pathway. Another sugar derived from D-fructose-6P, D-mannose, was also consistently observed as a by-product of D-glucose formation. D-Mannose likely accumulated as a result of the manZ gene deletion in all the strains studied, which prevented the uptake of not only D-glucose but also this sugar.

Of the several knockouts examined to clarify the route for D-glucose formation from the intermediate D-fructose-6P, only a deletion of the pgi gene coding phosphoglucose isomerase eliminated D-glucose formation. Although this result implicates D-glucose-6P as the direct precursor to extracellular D-glucose, we do not establish how D-glucose-6P itself is hydrolyzed. Unfortunately E. coli has numerous candidate enzymes that could hydrolyze D-glucose-6P: a periplasmic acid phosphatase (37), an alkaline phosphatase (23) and eight different haloacid dehalogenase-like hydrolases (27) have all been observed to hydrolyze D-glucose-6P under various environmental conditions. Each of these enzymes might mediate the final step to D-glucose during growth on D-xylose or L-arabinose.

All strains in this study had knockouts in the ptsG, manZ and glk genes encoding proteins involved in the principal means for D-glucose uptake in E. coli (10). There is no report of these proteins being involved in D-glucose export, and our results provide no guidance to this process. E. coli has several known porins and permeases that can translocate D-glucose through the outer and cytoplasmic membranes (though previous studies have invariably focused on sugar import). For example, porins OmpF and OmpC transport D-glucose by passive diffusion across the outer membrane (35, 36). LamB functions as a broad specificity glycoporin which transports D-glucose among other mono- and polysaccharides (30). Galactose permease (GalP) readily transports D-glucose across the cytoplasmic membrane (32). In fact, for strains deficient in the PTS uptake system (ptsH, ptsl, crr knockouts), GalP very effectively replaces the transport functions of the IICB^{Glc} PTS protein (19). Similarly, the periplasmic D-glucose/D-galactose binding receptor protein encoded by mglB binds D-glucose (25, 34) and contributes significantly to the growth and transport affinity for D-glucose at low extracellular D-glucose concentrations (24). Additionally, E. coli responds to knockouts in transport genes: the expression of the permeases galP, mglB and lamB increased as a result of a ptsHlcrr knockout (20). The mechanisms of D-glucose uptake under D-glucose-limiting and -excess conditions have been reviewed (18), and the various proteins involved in D-glucose uptake have recently been examined collectively in E. coli in the context of overflow metabolism and vaccine production (21). Interestingly, G1uP has been implicated in D-glucose export in Bacillus subtilis (33), but we found no similar E. coli protein. In our current study, any of these or other proteins could also be involved in D-glucose excretion.

A consistent result was that a greater yield of D-glucose was attained from L-arabinose than from D-xylose, and this difference must result from differences in the metabolism of these two pentoses by E. coli. Interestingly, D-xylose and L-arabinose are transported and enter the PP pathway through different routes in E. coli. D-Xylose is transported by several routes: a D-xylose/proton symporter (28), an ATP-binding dependent system (1) and by promiscuous transporter activity (26). The ATP-dependent system appears to predominate under normal growth conditions (22), indicating that D-xylose uptake generally demands energy directly in the form of ATP. Cellular options for the transport of L-arabinose similarly include a high affinity ATP-dependent system and a low affinity proton symport (11), as well as promiscuous transport (13). For this pentose the low affinity, ATP-independent system appears to predominate when both systems are present and the L-arabinose concentration is relatively high (11), although this process presumably affects availability of ATP also. After cellular uptake, both of these pentoses are ultimately converted to the common intermediate D-xylulose-5P (FIG. 2), through steps which require ATP for phosphorylation via D-xylulokinase or L-ribulokinase respectively for D-xylose or L-arabinose. Since the metabolism of these two pentoses would appear identical after D-xylulose, one might speculate that the difference in yield D-glucose yield between the two pentoses might be due to the difference in sugar transport mechanisms. Additional studies will have to clarify this difference.

If 2 moles of D-fructose-6P generated from 3 moles of D-xylose or L-arabinose (Eq. 2) are available for D-glucose formation (corresponding to 0.67 mol/mol), the theoretical D-glucose mass yield from either pentose is 0.80 g/g. This calculation considers the D-glyceraldehyde-3P generated from the flux-balanced PP pathway to be unavailable for D-glucose formation because additional D-glyceraldehyde-3P cannot re-enter the PP pathway without consuming D-fructose-6P. On the other hand, inclusion of the hypothetical conversion of D-glyceraldehyde-3P through the reverse Embden-Meyerhof-Parnas pathway to D-fructose-6P would result in a theoretical maximum yield 1.0 g/g. The greatest yield observed in the current study was about 0.3 g/g, a result probably due to the assimilation of some of the intermediate monosaccharides by other enzymes present in E. coli and not deleted in this study, and by the reversible enzymes in the PP pathway (transaldolase and transketolase) which would limit D-fructose-6P formation if these reactions approached equilibrium. Although not likely to serve as a process for generating the specific hexose D-glucose, this work demonstrates an approach to convert 5-carbon saccharides into 6-carbon saccharides, which could thereby both build carbon length and generate hexoses derived from D-fructose-6P not possible under typical conditions during growth on D-glucose.

Our results highlight two other aspects of metabolism in strains that have deletions in D-glucose uptake and other genes in upper metabolism. First, we detected no D-fructose as a product in any of the experiments, and our shake flask study with MEC 143 growing on D-fructose yielded only a low concentration of D-glucose. The absence of D-fructose in the (extracellular) medium is likely because E. coli uses a D-fructose-specific phosphotransferase system (fruA and fruB genes) and D-fructose-1P kinase (fruK) to metabolize D-fructose to D-fructose-1,6P₂, bypassing D-fructose-6P. In other words, assimilation of D-fructose via this route bypasses D-fructose-6P and would also prevent D-fructose accumulation in the strains studied. In contrast, once D-glucose is transported out of the cell, deletions of the ptsG and manZ genes prevent its uptake. A second noteworthy result from the mutants studied lies in the absence of D-glucose repression. For example, previous research has demonstrated that xylose isomerase is repressed in the presence of D-glucose (4). This effect appears to be caused specifically by D-glucose catabolite repression (8), an occurrence requiring the active catabolism of D-glucose, and which therefore is avoided in a strain unable to metabolize D-glucose. In our batch process accumulating nearly 5 g/L D-glucose (FIG. 3), we did not observe any deceleration of D-xylose utilization: the cells acted as though D-glucose was not present. So, E. coli is able to metabolize D-xylose in the presence of D-glucose when the D-glucose is not being metabolized.

In conclusion, D-glucose formation from either L-arabinose or D-xylose occurs as a result of the PP pathway leading to D-fructose-6P, which, unable to proceed into the glycolytic pathway due to a knockout in pfkA, equilibrates to D-glucose-6P. D-Glucose-6P likely hydrolyzes by one of several possible enzymes to D-glucose, which then accumulates when the cells are unable to metabolize it. We envision an analogous route could be used to generate similar sugars or sugar-containing compounds.

### References

1. Ahlem, C., W. Huisman, G. Heslund, and A. S. Dahms. 1982. Purification and properties of a periplasmic D-xylose-binding protein from Escherichia coli K-12. J. Biol. Chem. 257:2926-2931.

2. Baba, T., T. Ara, M. Hasegawa, Y. Takaki, Y. Okumura, M. Baba, K. A. Datsenko, M. Tomita, B. L. Wanner, and H. Mori. 2006. Construction of Escherichia coli K-12 in-frame, single-gene knockout mutants: the Keio collection. Mol. Syst. Biol. 1-11.

3. Bagno, A., F. Rastrelli, and G. Saielli. 2007. Prediction of the ¹H and ¹³C NMR spectra of □-D-glucose in water by DFT methods and MD simulations. Journal of Organic Chemistry. 72: 7373-7381.

4. Batt, C. A., M. S. Bodis, S. K. Picataggio, M. C. Claps, S. Jamas, and A. J. Sinskey. 1985. Analysis of xylose operon regulation by Mud (Apr, lac) fusion: trans effect of plasmid coded xylose operon. Can. J. Microbiol. 31:930-933.

5. Battley, E. H. 1991. Calculation of the heat of growth of Escherichia coli K-12 on succinic acid. Biotechnol. Bioeng. 37(4):334-343.

6. Bertrand, A., and J.-C. Barbe. 2002. Formation of γ-gluconolactone in a wine-like model system. J. Sci. Food Agri. 82:1571-1573.

7. Bhosale, S. H., M. B. Rao, and V. V. Deshpande. 1996. Molecular and industrial aspects of glucose isomerase. Microbiol. Rev. 60:280-300.

8. Briggs, K., W. Lancashire, and B. Hartley. 1984. Molecular cloning, DNA structure and expression of the Escherichia coli D-xylose isomerase. EMBO J. 3:611-616.

9. Curatolo, W., L. J. Neuringer, D. Ruben, and R. Haberkorn. 1983. Two-dimensional J-Resolved ¹H-nuclear magnetic resonance spectroscopy of □,□-D-glucose at 500 MHz. Carbohydrate Research. 112: 197-300.

10. Curtis, S. J., and W. Epstein. 1975. Phosphorylation of D-glucose in Escherichia coli mutants defective in glucosephosphotransferase, mannosephosphotransferase, and glucokinase. J. Bacteriol. 122:1189-1199.

11. Daruwalla, K. R., A. T. Paxton, and P. J. F. Henderson. 1981. Energization of the transport systems for arabinose and comparison with galactose transport in Escherichia coli, Biochem. J. 200:611-627.

12. Datsenko, K. A., and B. L. Wanner. 2000. One-step inactivation of chromosomal genes in Escherichia coli K-12 using PCR products. Proc. Natl. Acad. Sci. USA 97:6640-6645.

13. Desai, T. A., and C. V. Rao. 2010. Regulation of arabinose and xylose metabolism in Escherichia coli. Appl. Environ. Microbiol. 76:1524-1532.

14. Eiteman, M. A., and M. J. Chastain. 1997. Optimization of the ion-exchange analysis of organic acids from fermentation. Anal. Chim. Acta 338:69-75.

15. Eiteman, M. A., S. A. Lee, and E. Altman. 2008. A co-fermentation strategy to consume sugar mixtures effectively. J. Biol. Eng. 2:3.

16. Eiteman, M. A., S. A. Lee, R. Altman, and E. Altman. 2009. A substrate-selective co-fermentation strategy with Escherichia coli produces lactate by simultaneously consuming xylose and glucose. Biotechnol. Bioeng. 102:822-827.

17. Epting, K. L., C. Vieille, J. G. Zeikus, and R. M. Kelly. 2005. Influence of divalent cations on the structural thermostability and thermal inactivation kinetics of class II xylose isomerases. FEBS J. 272:1454-1464.

18. Ferenci, T. 1996. Adaptation to life at micromolar nutrient levels: the regulation of Escherichia coli glucose transport by endoinduction and cAMP. FEMS Microbiol. Rev. 18:301-317.

19. Flores, S., G. Gosset, N. Flores, A. A. Graaf, and F. Bolivar. 2002. Analysis of carbon metabolism in Escherichia coli Strains with an inactive phosphotransferase system by ¹³C labeling and NMR spectroscopy. Metabol. Eng. 4:124-137.

20. Flores, S., N. Flores, R. Anda, A. Gonzalez, A. Escalante, J. C. Sigala, G. Gosset, and F. Bolivar. 2005. Nutrient-scavenging stress response in an Escherichia coli strain lacking the phosphoenolpyruvate:carbohydrate phosphotransferase system, as explored by gene expression profile analysis. J. Mol. Microbiol. Biotechnol. 10:51-63.

21. Fuentes, L. G., A. R. Lara, L. M. Martinez, 0. T. Ramirez, A. Martinez, R. Bolivar, and G. Gosset. 2013. Modification of glucose import capacity in Escherichia coli: physiological consequences and utility for improving DNA vaccine production. Microb. Cell Fact. 12:42.

22. Hasona, A., T. Kim, F. G. Healy, L. O. Ingram, and K. T. Shanmugam. 2004. Pyruvate formate lyase and acetate kinase are essential for anaerobic growth of Escherichia coli on xylose. J. Bacteriol. 186:7593-7600.

23. Heppel, L. A., D. R. Harkness, and R. J. Hilmoe. 1962. A study of the substrate specificity and other properties of the alkaline phosphatase of Escherichia coli. J. Biol. Chem. 237:841-846.

24. Hua, Q., C. Yang, T. Oshima, H. Mori, and K. Shimizu. 2004. Analysis of gene expression in Escherichia coli in response to changes of growth-limiting nutrient in chemostat cultures. Appl. Environ. Microbiol. 70:2354 -2366.

25. Kalckar, H. M. 1971. The periplasmic galactose-binding protein of Escherichia coli. Science 174:557-565.

26. Khankal, R., J. W. Chin, and P. C. Cirino. 2008. Role of xylose transporters in xylitol production from engineered Escherichia coli. J. Biotechnol. 134:246-252.

27. Kuznetsova E., M. Proudfoot, C. F. Gonzalez, G. Brown, M. V. Omelchenko, I. Borozan, L. Carmel, Y. I. Wolf, H. Mori, A. V. Savchenko, C. H. Arrowsmith, E. V. Koonin, A. M. Edwards, A. F. Yakunin. 2006. Genome-wide analysis of substrate specificities of the Escherichia coli haloacid dehydrogenase-like phosphatase family. J. Biol. Chem. 281:36149-36161.

28. Lam, V. M., K. R. Daruwalla, P. J. Henderson, and M. C. Jones-Mortimer. 1980. Proton-linked D-xylose transport in Escherichia coli. J. Bacteriol. 143:396-402.

29. Liu, S. Y., J. Wiegel, and F. C. Gherardini. 1996. Purification and cloning of a thermostable xylose (glucose) isomerase with an acidic pH optimum from Thermoanaerobacterium strain JW/SL-YS 489. J. Bacteriol. 178:5938-5945.

30. Luckey, M., and H. Nikaido. 1980. Specificity of diffusion channels produced by 1-phase receptor protein of Escherichia coli. Proc. Natl. Acad. Sci. 77:167-171.

31. Matsushita, K., J. C. Arents, R. Bader, M. Yamada, O. Adachi, and P. W. Postma. 1997. Escherichia coli is unable to produce pyrroloquinoline quinone (PQQ). Microbiol. 143:3149-3156.

32. McDonald, T. P., A. R. Walmsley, and P. J. F. Henderson. 1997. Asparagine 394 in putative helix 11 of the galactose-H+ symport protein (GalP) from Escherichia coli is associated with the internal binding site for cytochalasin B and sugar. J. Biol. Chem. 272:15189-15199.

33. Mesak, L. R., F. M. Mesak, and M. K. Dahl. 2004. Expression of a novel gene, gluP, is essential for normal Bacillus subtilis cell division and contributes to glucose export. BMC Microbiol. 4:13.

34. Miller, D. M. 3^{rd}, J. S. Olson, and F. A. Quiocho. 1980. The mechanism of sugar binding to the periplasmic receptor for galactose chemotaxis and transport in Escherichia coli. J. Biol. Chem. 255:2465-2471.

35. Nikaido, H., and T. Nakae. 1979. The outer membrane of Gram-negative bacteria. Adv. Microbial Physiol. 20:163-250.

36. Nikaido, H., and E. Y. Rosenberg. 1983. Porin Channels in Escherichia coli: Studies with liposomes reconstituted from purified proteins. J. Bacteriol. 153:241-252.

37. Passariello, C., C. Forleo, V. Micheli, S. Schippa, R. Leone, S. Mangani, M. C. Thaller, and G. M. Rossonlini. 2006. Biochemicalcharacterization of the class B acid phosphatase (AphA) of Escherichia coli MG1655. Biochim. Biophys. Acta 1764:13-19.

38. Peng, F., J. L. Ren, F. Xu, J. Bian, P. Peng, and R. C. Sun. 2010. Fractionation of alkali-solubilized hemicelluloses from delignified Populus Gansuensis: Structure and Properties. J. Agric. Food Chem. 58:5743-5750.

39. Pfeffer, P. E., K. M. Valentine, and F. W. Parrish. 1979. Deuterium-induced differential isotope shift ¹³C NMR. 1. Resonance reassignments of mono- and disaccharides. J. Amer. Chem. Soc. 101:1265-1274.

40. Rossi, C., N. Marchettini, A. Donati, D. Medaglini, M. Valassina, S. Bastianoni, and E. Cresta. 1995. ¹³C-NMR determination of simultaneous xylose and glucose fermentation by a newly isolated strain (G11) of Klebsiella planticola. Biomass Bioenergy. 8:197-202.

41. Sapunova, L. I., A. G. Lobanok, I. O. Kazakevich, E. A. Shlyakhotko, and A. N. Evtushenkov. 2006. Biosynthetic features and properties of xylose isomerases from Arthrobacter nicotianae, Escherichia coli, and Erwinia carotovora subsp. atroseptica. Appl. Biochem. Microbiol. (Russ.) 42:246-251.

42. Sebastian, J., and C. Asensio. 1972. Purification and properties of the mannokinase from Escherichia coli. Arch. Biochem. Biophys. 151:227-233.

43. Ulrich, E. L., H. Akutsu, J. F. Doreleijers, Y. Harano, Y. E. Ioannidis, J. Lin, M. Livny, S. Mading, D. Maziuk, Z. Miller, E. Nakatani, C. F. Schulte, D. E. Tolmie, R. K. Wenger, H. Yao, and J. L. Markley. 2007. BioMagResBank. Nucleic Acids Res. 36:D402-D408.

44. U.S. EPA. 1983. Nitrogen, Ammonia. Method 250.1 (Colorimetric). pp. 350-1.1 - 350-1.4. In Methods for Chemical Analysis of Water and Wastes. EPA-600/4-79-020. U.S. E. P. A., Cincinnati, Ohio USA.

45. van Schie, B. J., K. J. Hellingwerf, J. P. van Kijken, M. G. L. Elferink, J. M. van Dijl, J. G. Kuenen, and W. N. Konings. 1985. Energy transduction by electron transfer via a pyrrolo-quinoline quinone-dependent glucose dehydrogenase in Escherichia coli, Pseudomonas aeruginosa, and Acinetobacter calcoaceticus (var. lwoffi). J. Bacteriol. 163:493-499.

46. Voronovsky, A. Y., O. B. Ryabova, O. V. Verba, O. P. Ishchuk, K. V. Dmytruk, and A. A. Sibirny. 2005. Expression of xylA genes encoding xylose isomerases from Escherichia coli and Streptomyces coelicolor in the methylotrophic yeast Hansenula polymorpha. FEMS Yeast Res. 5:1055-1062.

47. Wishart, D. S., C. G. Bigam, J. Yao, F. Abildgaard, H. J. Dyson, E. Oldfield, J. L. Markley, and B. D. Sykes. 1995. ¹H, ¹³C and ¹⁵N chemical shift referencing in biomolecular NMR. J. Biomol. NMR. 6:135-140.

48. Xia, T., M. A. Eiteman, and E. Altman. 2012. Simultaneous utilization of glucose, xylose and arabinose in the presence of acetate by a consortium of Escherichia coli strains. Microb. Cell Fact. 11:77.

Selected methods and materials for use in the present invention are also described in U.S. Pat. No. 8,551,758.

### Example 2

### Enhancing Glucose Accumulation

Glucose Formation. A clear understanding of how glucose is formed is important for improving intracellular and extracellular glucose formation as well as assisting the formation of other compounds. One strategy is to knockout specific genes encoding for enzymes hypothesized to be relevant to glucose formation. In particular, the mak gene encoding mannose kinase (FIG. 1 [K]), the xylA gene encoding xylose isomerase which is also a glucose isomerase (FIG. 1 [J]), the agp gene encoding glucose 1-phosphatase (FIG. 1[B]), and the pgm gene encoding phosphoglucomutase (FIG. 1 [C]) are expected to be involved in glucose formation. Strains with these knockouts (individually or in various combinations) can be studied for growth on xylose, arabinose or glycerol for glucose formation. If absence of glucose or curtailment of growth altogether is observed, this suggests an important step in glucose formation, which can be confirmed by overexpressing that enzyme in the mutant to restore growth and glucose formation. Intracellular metabolites that can be measured and that may accumulate include glucose-1P (Fu et al., 2000), glucose-6P (Lang and Michal, 1974; Doiron et al., 1994; Fu et al., 2000), UDP-glucose (Keppler and Decker, 1974; Nakai et al., 1999), glucose (Takahashi et al., 1995), and fructose-6P (Georgi et al., 2005). Glucose Transport. Our research shows that glucose can be exported from the cell. An understanding of glucose transport can be advanced by knocking out suspected permeases and transporters including galP (encoding galactose permease). Determination of intracellular glucose will be helpful in identifying proteins that play a role in glucose export. Knocking out a key exporter is expected to lead to increased intracellular glucose and/or curtailment of growth. Overexpression of identified transport proteins may enhance the extracellular accumulation of glucose.

Insights to both the sugar formation pathway and transport process can be obtained by completing DNA microarrays and looking for upregulated genes which indicate probable routes. Cultures for microarray analysis are grown using chemostat cultures (under steady-state conditions). Strains which do not accumulate or transport glucose (for example, by not having the pfkA knockout) can serve as useful controls.

Improving Glucose Formation. Glucose formation can be improved by a combination of knockouts and process strategies. Several additional glucose-metabolizing genes are candidates for knockout, including gcd encoding glucose dehydrogenase (EC 1.1.5.2) and key genes of the Entner-Doudoroff pathway. We also anticipate that glucose formation can be affected by growth strategy. For example, growing under conditions in which the carbon source (such as xylose) is in excess and growth is limited by another nutrient (N, P, etc.) is expected to increase the yield of hexose formation by directing the most carbon to products and the least to biomass. Growing strains in chemostats under phosphorus-limiting conditions is particularly envisioned because an important step in glucose formation (and other hexoses) is expected to be the dephosphorylation of some sugar-phosphate:

glucose-P glucose+Pi [6]

From the standpoint of mass-action, one might hypothesize that limiting the product phosphate might drive the process forward to glucose formation.

Finally, we expect that co-metabolism strategies can promote sugar formation. For example, we have previously used this strategy to make E. coli auxotrophic for acetate by knocking out pyruvate dehydrogenase genes (Zhu et al., 2008). Such a strain requires both acetate and glucose (or another sugar) for growth, and therefore either one can serve as a growth-limiting substrates. Then, growing with excess glucose under acetate-limiting (fed-batch) conditions, E. coli will accumulate 90 g/L pyruvate in less than 40 h. A similar strategy can be applied to monosaccharide formation by decoupling the TCA cycle from glycolysis and growing under (for example) acetate-limiting conditions with excess xylose.

Other Sugar Transformations. The basic strategy for hexose formation relies on knocking out a specific sugar uptake system and then diverting carbon to that unmetabolizable sugar. This can be applied to the conversion of fructose to glucose and glucose to fructose. Briefly, to illustrate the approach, for the conversion of fructose to glucose glucose consumption can be eliminated (as already shown), and then fructose utilization can be curtailed, for example by knocking out one or more of several genes: pfkA and zwf as above, mak (mannokinase, FIG. 1[K]),fruK (fructose-1P kinase , FIG. 1[M]), fbaA/bfaB (fructosbisphophate aldolase, FIG. 1[P]), and talA/talB (transaldolase, FIG. 1[I]). Some of these knockouts might require supplementing the medium with a secondary carbon source such as glycerol or acetate, which can be used as growth-limiting substrates as described above. Similarly, for the conversion of glucose to fructose, the fructose PTS system (levF levG fruA) can be eliminated, and more glucose can be directed to fructose: knockouts in zwf and pfkA as above, as well as talA/talB; and other genes such as xylA or agp.

### Example 3

### Incorporation of 1-Carbon Compounds into Glucose and Fructose

The Ru5P cycle is a pathway used by several organisms to incorporate methane via methanol and formaldehyde into central metabolism. The soluble alcohol methanol is oxidized to formaldehyde by the enzyme methanol dehydrogenase (EC 1.1.1.244). The soluble NAD-dependent methanol dehydrogenase from Bacillus methanolicus, which has been previously expressed successfully in Escherichia coli (de Vries et al. 1992), is a suitable enzyme.

The incorporation of one-carbon compounds into metabolism via the Ru5P cycle depends on the activity of two enzymes: HPS combines D-ribulose-5P and formaldehyde while the enzyme HI interconverts hexulose-6P and D-fructose-6P. In addition to methanol dehydrogenase, these two enzymes can be overexpressed in a glucose-forming strain.

A methanol-arabinose or methanol-xylose mixture can be used as a carbon source, and enhanced glucose formation is expected. Previous studies have shown that E. coli tolerates 30 g/L methanol with minimal growth inhibition (Ganske and Bornscheuer, 2006), so we do not anticipate difficulty in implementing a feeding strategy. Additionally, ribulose-5P 3-epimerase (FIG. 1[G]) can be overexpressed to ensure ample supply of D-ribulose-5P as the substrate for HPS. Similar to the process strategy described above for enhanced glucose formation, limiting growth by another nutrient such as nitrogen, phosphorus or even another carbon-source such as acetate can be a means to elevate the conversion efficiency. It is expected that methanol can thereby be incorporated into a hexose.

### Example 4

### Formation of Products from Glucose and Other Sugars

With bacterial cells that are able to accumulate glucose and other sugars, several products become feasible that would not have normally been considered via microbial processes. To demonstrate and explore the possible benefits of sugar accumulation, we will examine the formation of three classes of compounds detailed below.

A. D-Glucose-δ-lactone/D-Gluconate

D-Glucose-δ-lactone (G-lactone) is readily formed from glucose by soluble glucose dehydrogenase (GDH, EC 1.1.1.47) using either NAD+ or NADP+ (FIG. 7[A]). G-lactone is of interest as a substrate for the formation of biodegradable polyesters (Tsutsumi et al., 2004).

GDH has been used in biocatalysis to regenerate the cofactor NADPH during enzyme reduction (Xu et al., 2006, 2007), and also in co-expression systems in which elevated NADPH is desired (Zhang et al., 2011). G-lactone is readily hydrolyzed to D-gluconate by gluconolactonase (EC 3.1.1.17, FIG. 7[B]). Although E. coli is thought to have an active gluconolactonase (Hucho and Wallenfels, 1972), an encoding gene has surprisingly not been identified. G-lactone hydrolysis is also known to occur abiotically via a first order hydrolysis (Koga et al., 1967). D-Gluconate formed can be phosphorylated to D-gluconate-6P by gluconokinases (EC 2.7.1.12, FIG. 7[C]) encoded by gntK and idnK. In a parallel pathway involving the phosphorylated compounds, G-lactone-6P is hydrolyzed both by 6P-gluconolactonase (FIG. 7[D]) and spontaneously (but poorly) to D-gluconate-6P (Kupor and Fraenkel, 1969, 1972), a compound which marks the start of the Entner-Doudoroff pathway.

We will examine the formation of G-lactone and D-gluconate in E. coli by expressing GDH from Bacillus subtilis (Zhang et al., 2011) in the glucose-generating strains, and growing cells under growth-limiting conditions (with excess carbon) as elaborated above. We anticipate that additionally the gntK and idnK genes (FIG. 7[C]) may be knocked out to prevent significant D-gluconate loss through the Entner-Doudoroff pathway. By measuring both G-lactone and D-gluconate concentrations (which we have already achieved by HPLC), our research will clarify the mechanism of G-lactone hydrolysis in E. coli. We hope to identify whether E. coli indeed does have a gluconolactonase: for example, a knockout in the relatively recently identified pgl gene encoding 6P-gluconolactonase (Thomason et al., 2004, FIG. 7[D]) will demonstrate whether this enzyme also participates in the hydrolysis of G-lactone to D-gluconate. The formation of D-gluconate involves the formation of NADH, and therefore we anticipate that the generation of D-gluconate (compared to glucose formation) will generate more ATP for the cell under aerobic conditions and thus greater biomass yield.

Commercially competitive D-gluconate formation by E. coli is not the goal: Gluconobacter spp. are known to form D-gluconate (Humphrey and Reilly, 1965; Koga et al., 1967). Instead, this brief and relatively straightforward, initial study on the formation of G-lactone and D-gluconate—involving one transformation using a well-studied enzyme and about 3 knockouts—will instead provide us with important information on how to generate products directly from a hexose. For example, is there any benefit from blocking glucose export to facilitate product formation (without affecting other aspects of the process)? How does nitrogen- or phosphorus-limited growth impact recombinant gene expression and product formation?

### B. Glycosylation via UDP-glucose

As detailed in a previous section, the formation of a very wide range of glucosides by the process of glycosylation specifically involves the donation of a monosaccharide unit (e.g., glucose) via a glyconucleotide (e.g., UDP-glucose). A common presumption in previous research is that ample glucose-1P, the precursor to UDP-glucose, is available to support the formation of UDP-glucose (and researchers have sought to ensure UDP-glucose availability, for example, by adding a great excess of UDP-glucose directly into the medium). Our underlying hypothesis is that an elevated in vivo concentration of glucose-1P, uniquely made possible by this proposed research, and overexpression of UTP-glucose-1P uridyltransferase forming UDP-glucose will promote glucoside formation. Our strategy is to feed in the aglycon into a growing culture in the presence an appropriate glycosyltransferase and generate the glycosylated product.

The process of UDP-glucose formation may be facilitated by redirecting glucose flux to glucose-1P (FIG. 1). We will overexpress native phosphoglucomutase (pgm, FIG. 1[C]) and knockout glucose 1-phosphatase (agp, FIG. 1[B]) to direct D-glucose-6P to D-glucose-1P.

Appropriate additional knockouts will be made to compare xylose and fructose as carbon sources. (For example, to direct xylose more to glucose-1P, we envision knocking out mak, FIG. 1[K].) For the formation of UDP-glucose from glucose-1P, we will overexpress galF encoding UTP-glucose-1P uridyltransferase (EC 2.7.7.9, FIG. 6[A]). Appropriate control strains will also be generated so that we can draw conclusions regarding the limiting step in UDP-glucose formation. We will measure intracellular concentrations of UDP and UDP-glucose (Keppler and Decker, 1974; Nakai et al., 1999).

The key step in glycosylation is the glycosyltransferase (GT), the enzyme which transfers the monosaccharide to an aglycon to form the glucoside. As noted above, tens of thousands of enzymes are known from which we can choose, and initially we will select a few of particular interest.

First, we will examine the formation of the anthocyanin cyanidin 3-O-glucoside (also known as chrysanthemin) from the anthocyanidin cyanidin (FIG. 6) using UDP-glyosyltransferase VL3GT from Vitis labrusca (Concord grape, Hall et al., 2012). Anthocyanins are members of a large and diverse group of plant secondary metabolites, and a growing body of evidence suggests some anthocyanins possess medicinal properties. VL3GT has been sequenced and expressed in E. coli and preferentially glucosylates cyanidin at the 3-position. Standards for both cyanidin and cyanidin 3-glucoside are available commercially. We plan to grow our glucose-1P generating strain and express synthetic VL3GT which has been codon optimized for E. coli. We will feed in cyanidin and measure the appearance of cyanidin 3-glucoside. The enzyme shows high affinity (KM=4.8 μM) for cyanidin (Hall et al., 2012). If expression of this protein is poor, we are able to co-express groES-groEL chaperone proteins.

Second, UGT85K4 and UGT85K5 isolated from cassava are fairly broad GTs demonstrated to glycosylate simple alcohols such as 2-propanol and 2-butanol as well as hydroxynitriles such as mandelonitrile and p-hydroxymandelonitrile and isoflavonoids daidzein and genistein (Kannangara et al., 2011). Similarly as for cyanidin, after expressing each of these GTs individually in our strains, we will feed in small alcohols and hydroxynitriles and examine the culture for glycosylated products.

Third, OleD is the oleandomycin GT from Streptomyces antibioticus that has been extensively studied in enzyme evolution studies to increase the enzyme's promiscuity (Gantt et al., 2008; Williams et al., 2011). The ASP variant readily accomplishes O-glycosylation, N-glycosylation and S-glycosylation (Gantt et al., 2008). Analogous to the process for the two previous enzymes studied, we will overexpress OleD-ASP or other variants in our strains, and feed into the culture substrates exemplifying each type of glycosylation: phenol, thiophenol and aniline. Because the enzyme can catalyze iterative glycosylation, we anticipate multiple products (e.g., glucosyl-glucoside) depending on the feed rate and availability of the aglycon.

Because a wide range of GTs are available and scores of labs are studying GTs, we anticipate our ideas will lead to collaborations with other researchers having an interest in a specific product or GT. Our overall goal is not to focus on a specific product, as the products listed above are merely illustrative of a research plan. Rather, our overall goal is to study generally the formation of glycosylated compounds derived in cultures accumulating glucose/UDP-glucose.

Although our focus will be UDP-glucose, several other glyconucleotides are adjacent enzymatically to UDP-glucose. For example, in E. coli UDP-galactose is formed via a 4-epimerase (EC 5.1.3.2) encoded by galE (Eq. 7A) or a transferase (EC 2.7.7.12) encoded by galT (Eq. 7B):

UDP-glucose→UDP-galactose [7A]

UDP-glucose+galactose-1P→UDP-galactose+glucose-1P [7B]

while UDP-glucuronate is formed via UDP-glucose 6-dehydrogenase (EC 1.1.1.22) encoded by ugd:

UDP-glucose+2NAD→UDP-glucuronate+2NADH [8]

The latter process (Eq. 8) would appear to be particularly feasible under aerobic conditions as a result of the increased availability of NADH.

Depending on our success with forming glucosides as described above, we may explore the formation of glucuronate or galactose-glycosylated compounds, for example by overexpression galE or ugd and by using transferases more specific to UDP-glucuronate, etc.

One potential challenge is that the aglycons and the glycosylated product will poorly transport across the cell membrane. Previous research has surprisingly shown, however, that large aglycons such as 4-methylumbelliferonecoumarin, novobiocic acid, quercetin, resveratrol and daidzein (all polyaromatics) and their corresponding glucosides do indeed transport readily (Lim et al., 2006; Williams, et al., 2011), so we do not anticipate this problem will occur. Nevertheless, as described above we plan to examine a wide range of compounds (including readily permeable small alcohols) so that we understand the limitations that this proposed process has for glucoside formation and whether substrate/product transport is in some cases an issue.

### C. L-Gulose

As just one example of a rare sugar, L-gulose is a component of antiviral medications (for

HIV and HBV) including Epivir (Glaxo Smith Kline), Emtriva (Gilead) and Pentacept (Pharmasset) (Chu et al., 2001; Jeong et al., 1993), and the potent anticancer compound bleomycin formed by Streptomyces verticillus (Sugiura et al., 1983). Unfortunately, L-gulose accumulates minimally in nature, and therefore this sugar is extremely costly (˜$1000/g) and in inadequate supply. Current methods to form L-gulose are based on the bioconversion (i.e., without metabolism) of D-sorbitol to L-gulose using heterologous mannitol-l-dehydrogenase (ManDH) by growing E. coli on glycerol in complex medium (Woodyer et al., 2010), resulting in low productivity.

We will examine an alternate means of L-gulose synthesis by E. coli using D-xylose or D-glucose as the sole carbon source in a defined medium (FIG. 8). (For either carbon source, we will be using a strain which is curtailed in its ability to consume D-fructose.) Specifically, we will transform cells with NAD-dependent sorbitol dehydrogenase (SDH, FIG. 8[A]) from Rhodobacter sphaeroides (SDH, EC 1.1.1.14) and NADP-dependent ManDH (FIG. 8[B]) from Apium graveolens. Numerous SDHs exist, and they are often referred to as aldose reductase when a sugar is the substrate. Generally, NADPH-dependent SDHs (EC 1.1.1.21) have wide substrate specificity, such as the enzymes from Homo sapiens and Rattus norvegicus, which are particularly well-studied in the context of their role in cardiovascular disease (Kakeno et al., 2005). However, these SDHs would likely not be suitable because they also act on other sugars, such as D-xylose which results in D-xylitol. On the other hand, NAD-dependent SDHs (FIG. 8[A]) are preferred for our work because of their narrower substrate specificity. For example, SDH from R. sphaeroides (Schauder et al., 1995) or Pseudomonas spp. (Schneider and Giffhorn, 1991) selectively interconverts D-fructose and D-sorbitol, with the reduction of D-fructose optimal at a pH near 7 and the oxidation of D-sorbitol optimal at a pH of 9-11. For the second enzyme in the pathway, the unique NADP-dependent ManDH from Apium graveolens (FIG. 8[B]) will be used because it is regioselective at the 1 position and stereoselective at the 2 position, allowing it to catalyze the conversion of D-sorbitol to L-gulose (Stoop et al., 1996).

Although we plan to form L-gulose in strains that are deficient in the transport of D-glucose, we also may have to be concerned about the transport and utilization of the intermediate D-sorbitol. D-Sorbitol consumption by the sorbitol-pts system can be eliminated by knocking out the srlB, srlE and srlA genes.

### References

Allen, J. R., Harris, C. R., Danishefsky, S. J. (2001) Pursuit of optimal carbohydrate-based anticaner vaccines: preparation of a multiantigenic unimolecular glycopeptide containing the Tn, MBrl and Lewis^{y} Antigens. J Amer. Chem. Soc. 123, 1890-1897.

Alper, H., and Stpehanopoulos, G. (2009) Engineering for biofuels: exploiting innate microbial capacity or importing biosynthetic potential? Nat Rev Microbiol 7, 715-723.

Banerjee, S., Mudliar, S., Sen, R., Giri, B., Satpute, D., Chakrabarti, T., and Pandey, R. A. (2010) Commercializaing lignocellulosic biotechnoal: technology bottlenecks and possible remedies. Biofuels Bioprod Bioref 4, 77-93.

Bartolozzi, A. and Seeberger, P. H. (2001) New approaches to the chemical synthesis of bioactive oligosaccharides. Curr Opin Struct Biol 11, 587-592.

Beerens, K., Desmet, T., and Soetaert, W. (2012) Enzymes for the biocatalytic production of rare sugars. J Ind Microbiol Biotechnol 39, 823-834.

Bruant, G., Lévesque, M.-J., Peter, C., Guiot, S. R., and Masson, L. (2010) Genomic analysis of carbon monoxide utilization and butanol production by Clostridium carboxidivorans strain P7T. PLoS one 5, 9.

Chakraborty, S., Zhang, J., Krause, J. A., and Guan, H. (2010) An efficient nickel catalyst for the reduction of carbon dioxide with a borane. J Amer Chem Soc 132, 8872-8873.

Chu, C. K., Cheng, Y.-C., and Qu, F. (2001) L-b-dioxalane uridine analogs and their pharmaceutical compositions. U.S. Pat. No. 6,274,589.

Curtis, S. J., and Epstein, W. (1975) Phosphorylation of D-glucose in Escherichia coli mutants defective in glucosephosphotransferase, mannosephosphotransferase, and glucokinase. J Bacteriol 122, 1189-1199.

de Vries, G. E., Arfman, N., Terpstra, P., and Dijkhuizen, L. (1992) Cloning, expression, and sequence analysis of the Bacillus methanolicus Cl methanol dehydrogenase gene. J Bacteriol 174, 5346-5353.

Dedhia, N. N., Hottiger, T., and Bailey, J. E. (1994) Overproduction of glycogen in Escherichia coli blocked in the acetate pathway improves cell growth. Biotechnol Bioeng 44, 132-139.

Doiron, B., Cuif, M.-H., Kahn, A., and Diaz-Guerra, M.-J. M. (1994) Respective roles of glucose, fructose, and insulin in the regulation of the liver-specific pyruvate kinase gene promoter. J Biol Chem 269, 10213-10216.

Dondoni, A., Marra, A., and Massi, A. (1997) Carbohydrate homologation by the use of 2-(trimethylsilyl)thiazole. Preparative scale synthesis of rare sugars: L-gulose, L-idose, and the disaccharide subunit of bleomycin A(2). J Org Chem 62, 6261-6267.

Eiteman, M. A., Lee, S. A. and Altman, E. (2008) A Co-Fermentation Strategy to Consume Sugar Mixtures Effectively. J Biol Eng. 2:3.

Flint, J., Taylor, E., Yang, M., Bolam, D. N., Tailford, L. E., Martinez-Fleites, C., Dodson, E. J., Davis, B. G., Gilbert, H. J., and Davies, G. J. 2005. Structural dissection and high-throughput screening of mannosylglycerate synthase. Nat Struct Molec Biol. 12, 608-614.

Flores, N., Xiao, J., Berry, A., Bolivar, F., and Valle, F. (1996) Pathway engineering for the production of aromatic compounds in Escherichia coli. Nat Biotech 14, 620-623.

Foti, R. S., and Fisher, M. B. (2012) UDP-glucuronosyltransferases: pharmacogenetics, functional characterization, and clinical relevance. IN: Encyclopedia of Drug Metabolism and Interactions.

Fu, L., Miseta, A., Hunton, D. Marchase, R. B., and Bedwell, D. M. (2000) Loss of the major isoform of phosphoglucomutase results in altered calcium homeostasis in Saccharomyces cerevisiae. J Biol Chem 275, 5431-5440.

Ganske, F., and Bornscheuer, U. T. (2006) Growth of Escherichia coli, Pichia pastoris and Bacillus cereus in the presence of the ionic liquids [BIM][BF₄] and [BMIM][PF₆] and organic solvents. Biotechnol Lett 28, 465-469.

Gantt, R. W., Goff, R. D., Williams, G. J., and Thorson, J. S. (2008) Probing the aglycon promiscuoity of an enginered glycosyltransferase Ange Chem Intl Ed 47, 8889-8892.

Georgi, T., Rittmann, D., and Wendisch, V. F. (2005) Lysine and glutamate production by Corynebacterium glutamicum on glucose, fructose and sucrose: roles of malic enzyme and fructose-1,6-bisphosphatase. Metabol Eng 7, 291-301.

Hall, D., Yuan, X. X., Murata, J., and De Luca, V. (2012) Molecular cloning and biochemical characterization of the UDP-glucose:flavonoid 3-o-glucosyltransferase from Concord grape (Vitis labrusca). Phytochem 74:90-99.

Herter, S., Farfsing, J., Gad'On, N., Rieder, C., Eisenreich, W., Bacher, A., Fuchs, G. (2001) Autotrphic CO₂ fixation by Chloroflexus aurantiacus: study of glyoxylate formation and assimilation via the 3-hydroxypropionate cycle. J Bacteriol 183, 4305-4316.

Hofmann, C., Boll, R., Heitmann, B., Hauser, G., Durr, C., Frerich, A., Weitnauer, G., Glaser, S. J., Bechthold, A. (2005) Genes encoding enzymes responsible for biosynthesis of L-lyxose and attachment of eurekanate during avilamycin biosynthesis. Chem Biol 12, 1137-1143.

Hotze, M., Schroder, G., and Schroder, J. (1995) Cinnamate 4-hydrxylase from Catharanthus roseus, and a strategy for the function expression of plant cytochrome P450 proteins as translational fusions with P450 reductase in Escherichia coli. FEBS Lett 374, 345-350.

Huber, F., and Erni B. (1996) Membrane topology of the mannose transporter of Escherichia coli K12. Eur JBiochem 239, 810-817.

Hucho, F., and Wallenfels, K. (1972) Glucono-d-lactonase from Escherichia coli. Biochim Biophys Acta 276, 176-179.

Humphrey, A. E., and Reilly, P. J. (1965) Kinetics studies of gluconic acid fermentation. Biotechnol Bioeng 7, 229-244.

Hwang, E. I., Kaneko, M., Ohnishi, Y., Horinouchi, S. (2003) Production of plant-specific flavanones by Escherichia coli containing an articificial gene cluster. Appl Environ Microbiol 69, 2699-2706.

Jahn, U., Huber, H., Eisenreich, W., Hugler, M., Fuchs, G. (2007) Insights into the autotrophic CO₂ fixation pathway of the Archaeon Ignicoccus hospitalis: comprehensive analysis of the central carbon metabolism. J Bacteriol 189, 4108-4119.

Jeong, L. S., Schinazi, R. F., Beach, J. W., Kim, H. O., Nampalli, S., Shanmuganathan, K., Alves, A. J., McMillan, A., Chu, C. K., and R. J. Mathis, R. J. (1993) Asymmetric synthesis and biological evaluation of b-L-(2R,5S)-and a-L-(2R,5R)-1,3-oxathiolane-pyrimidine and -purine nucleosides as potential anti-HIV agents. J Med Chem 36, 181-195.

Kaneko, M., Bucciarelli, L., Hwang, Y. C., Lee, L., Yan, S. F., Schmidt, A. M., and Ramasamy, R. (2005) Aldose reductase and AGE-RAGE pathways: key players in myocardial ischemic injury. Ann NY Acad Sci 1043, 702-709.

Kannangara, R., Motawia, M. S., Hansen, N. K. K., Paquette, S. M., Olsen, C. E., Møller, B. L., and Jorgensen, K. (2011) Characterization and expression profile of two UDP-glucosyltransferases, UGT85K4 and UGT85K5, catalyzing the last step in cyanogenic glucoside biosynthesis in cassava. Plant J 68, 287-301.

Kato, N., Ohashi, H., Tani, Y., and Ogata, K. (1978) 3-Hexulosephosphate synthase from Methylomonas aminofaciens 77a. Purification, properties and kinetics. Biochim Biophys Acta 523, 236-244.

Kato, N., Yurimoto, H., and Thauer, R. K. (2006) The physiological role of the ribulose monophosphate pathway in bacteria and archaea. Biosci Biotechnol Biochem 70, 10-21.

Koeller, K. M., and Wong, C. H. (2000) Emerging themes in medicinal glycoscience. Nat Biotechnol 18, 835-841.

Koga, S., Burg, C. R., and Humphrey, A. E. (1967) Computer simulation of fermentation systems. Appl Microbiol 15, 683-689.

Koike, T., and Ikariya, T. (2004) Mechanistic aspects of formation of chiral ruthenium hydride complexes from 16-electron ruthenium amide complexes and formic acid: facile reversible decarboxylation and carboxylation. Adv Synth Catal 346, 37-41.

Kurumbang, N. P., Liou, K., and Sohng, J. K. (2010) Biosynthesis of paromamine derivatives in engineered Escherichia coli by heterologous expression. J Appl Microbiol 108, 1780-1788.

Kuper, S. R., and Fraenkel, D. G. (1969) 6-Phosphogluconolactonase mutants of Escherichia coli and a maltose blue gene. J Bacteriol 100, 1296-1301.

Kuper, S. R., and Fraenkel, D. G. (1972) Glucose metabolism in 6-phosphogluconolactonase mutants of Escherichia coli. J Biol Chem 247, 1904-1910.

Lerner, L. M. and Mennitt, G. (1994) A new synthesis of L-talose and preparation of its adenine nucleosides. Carbohydr Res 259, 191-200.

Lim, E.-K., Ashford, D. A. Hou, B., Jackson, R. G., and Bowles, D. J. (2004) iArabidopsis glycosyltransferases as biocatalysts in fermentation for regioselective synthesis of diverse quercetin glucosides. Biotechnol Bioeng 87, 623-631.

Lim, E.-K., Ashford, D. A., Bowles, D. J. (2006) The synthesis of amll-molecular rhamnosides through the rational design of a whole-cell biocatalysis system. ChemBioChem 7, 1181-1185.

Matsumura, H., Okumura, K., Shimamura, T., Ikenaga, N., Miyake, T., and Suzuki, T. (2006) Selective oxidation of methane to formaldehyde over antimony oxide-loaded catalyst. J Molec Catal A 250, 122-130.

Mitsui, R., Sakai, Y., Yasueda, H., and Kato, N. (2000) A novel operon encoding formaldehyde fixation: the ribulose monophosphate pathway in the gram-positive facultative methylotrophic bacterium Mycobacterium gastri MB 19. J Bacteriol 182, 944-948.

Nakai, T., Tonouchi, N., Kohishi, T., Kojima, Y., Tsuchida, T., Yoshinaga, F., Sakai, F., and Hayashi, T. (1999) Enhancement of cellulose production by expression of sucrose synthase in Acetobacter xylinum. Proc Natl Acad Sci 96, 14-18.

Ohnishi, Y.-Y., Matsunaga, T., Nakao, Y., Sato, H., and Sakaki, S. (2005) Ruthenium(II)-catalyzed hydrogenation of carbon dioxide to formic acid. Theoretical study of real catalyst, ligand effects, and solvation effects. J Amer Chem Soc 127, 2021-4032.

Postma, P. W., Lengeler, J. W., and Jacobson, G. R. (1993) Phosphoenolpyruvate: carbohydrate phosphotransferase systems of bacteria. Microbiol Rev 57, 543-594.

Sakai, Y., Mitsui, R., Katayama, Y., Yanase, H., and Kato, N. (1999) Organization of the genes involved in the ribulose monophosphate pathway in an obligate methylotrophic bacterium, Methylomonas aminofaciens 77a. FEMS Microbiol Lett 176, 125-130.

Schauder, S., Schneider, K.-H., and Giffhorn, F. (1995) Polyol metabolism of Rhodobacter sphaeroides: biochemical characterization of a short-chain sorbitol dehydrogenase. Microbiol 141, 1857-1863.

Schneider, K.-H., and Giffhorn, F. (1991) Sorbitol dehydrogenase from Pseudomonas sp.: purfication, characterization and application to quantitative determination of sorbitol. Enzyme Microb Technol 13, 332-337.

Seibold, G., Dempf, S., Schreiner, J., and Eikmanns, B. J. (2007) Glycogen formation in Corynebacterium glutamicum and role of ADP-glucose pyrophosphatase. Microbiol. 153, 1275-1285.

Shimamura, T., Okumura, K., Nakagawa, K., Ando, T., Ikenga, N., and Suzuki T. (2004) Direct formation of formaldehyde from methane and carbon dioxide over vanadium oxide catalysts. J Molecul Catal A 211, 97-102.

Simkhada, D., Lee, H. C., and Sohng, J. K. (2010) Genetic engineering approach for the production of rhamnosyl and allosyl flavonoids from Escherichia coli. Biotechnol Bioeng 107, 154-162.

Takahashi, N., Kalfas, S., and Yamada, T. (1995) Phosphorylating enzymes involved in glucose fermentaiton of Actinomyces naeslundii. J Bacteriol 177, 5806-5811.

Tianwei, M., Balacrishna, P. S., Lian, Z. Y., Sheng, L. J. Kirupa, S., Jinfa, D., Chunguang, W., Hongbum, K., and Gary, M. N. (1996) Structure-activity relationships of 1-(2-deoxy-2-fluoro-b-L-arabino-furanosyl) pyrimidine nucleosides as anti-hepatitis B virus agents. J Med Chem 39, 2835-2843.

Thomason, L. C., Court, D. L., Datta, A. R., Khanna, R., and Rosner, J. L. (2004) Identification of the Escherichia coli K-12 ybhE gene as pgl, encoding 6-phosphogluconolactonase. J Bacteriol 186, 8248-8253.

Thorson, J. S., Hosted, Jr., T. J., Jiang, J., Biggins, J. B., and Ahlert, J. (2001) Nature's carbohydrate chemists: The enzymatic glycosylation of bioactive bacterial metabolites. Curr Org Chem 5, 139-167.

Tochikura, T., Kawai, H., Tobe, S., Kawaguchi, K., Osugi, M., and Ogata, K. (1968) Studies on microbial metabolisms of sugar nucleotides: (I) UDP-glucose formation from 5;-UMP by yeasts. J Ferment Technol 46, 957-969.

Tsutsumi, N., Oya, M., and Sakai, S. (2004). Biodegradable network polyesters from glucolactone and citric acid. Macromol 27, 5971-5976.

Williams, G. J., Yang, J., Zhang, C., and Thorson, J. S. (2011) Recombinant E. coli prototype strains fro in vivo glycorandomization. ACS Chem Biol 6, 95-100.

Woodyer, R. D., Wymer, N. J., Racine, F. M., Khan, S. N., and Saha, B. C. (2008) Efficient Production of L-ribose with a recombinant Escherichia coli catalyst. Appl Environ Microbiol 74, 2967-2975.

Woodyer, R. D., Christ, T. N., and Deweese, K. A. (2010) Single-step bioconversion for the preparation of L-gulose ande L-galactose. Carbohyd Res 345, 363-368.

Xu, Z. N., Liu, Y., Fang, L. M., Jiang, X. X., Jing, K. J., and Cen, P. L. (2006) Construction of a two-strain system for asymmetric reduction of ethyl 4-chloro-3-oxobutanoate to (S)-4-chloro-3-hydroxybutanoate ethyl ester. Appl Microbiol Biotechnol 70, 40-46.

Xu, Z. N., Jing, K. J., Liu, Y., and Cen, P. L. (2007) High-level expression of recombinant glucose dehydrogenase and its application in NADPH regeneration. J Ind Microbiol Biotechnol 34, 83-90.

Yan, Y., Chemler, J., Huang, L., Martens, S., and Koffas, M. A. G. (2005) Metabolic engineering of anthocyanin biosynthesis in Escherichia coli. Appl Environ Microbiol 71, 3617-3623.

Yanase, H., Ikeyama, K., Mitsui, R., Ra, S., Kita, K., Sakai, Y., and Kato, N. (1996) Cloning and sequence analysis of the gene encoding 3-hexulose-6-phosphate synthase from the methylotrophic bacterium, Methylomonas aminofaciens 77a, and its expression in Escherichia coli. FEMS Microbiol Lett 135, 201-205.

Yasueda, H., Kawahara, Y., and Sugimoto, S.-I. (1999) Bacillus subtilis yckG and yckF encode two key enzymes of the ribulose monophosphate pathway used by methylotrphos, and yckH is required for their expression. J Bacteriol 181, 7154-7160.

Yu, H., and Stephanopoulos, G. (2008) Metabolic engineering of Escherichia coli for biosynthesis of hyaluronic acid. Metabol Eng 10, 24-32.

Zhang, J.-D., Li, A.-T., Yu, H.-L., Imanaka, T., and Xu, J.-H. (2011) Synthesis of optically pure S-sulfoxide by Escherichia coli transformant cells coexpressing the P450 monooxygenase and glucose dehydrogenase genes. J Ind Microbiol Biotechnol 38, 633-641.

### Example 5

### Formation of Glucose and Glucose-6P Metabolites from Pentoses

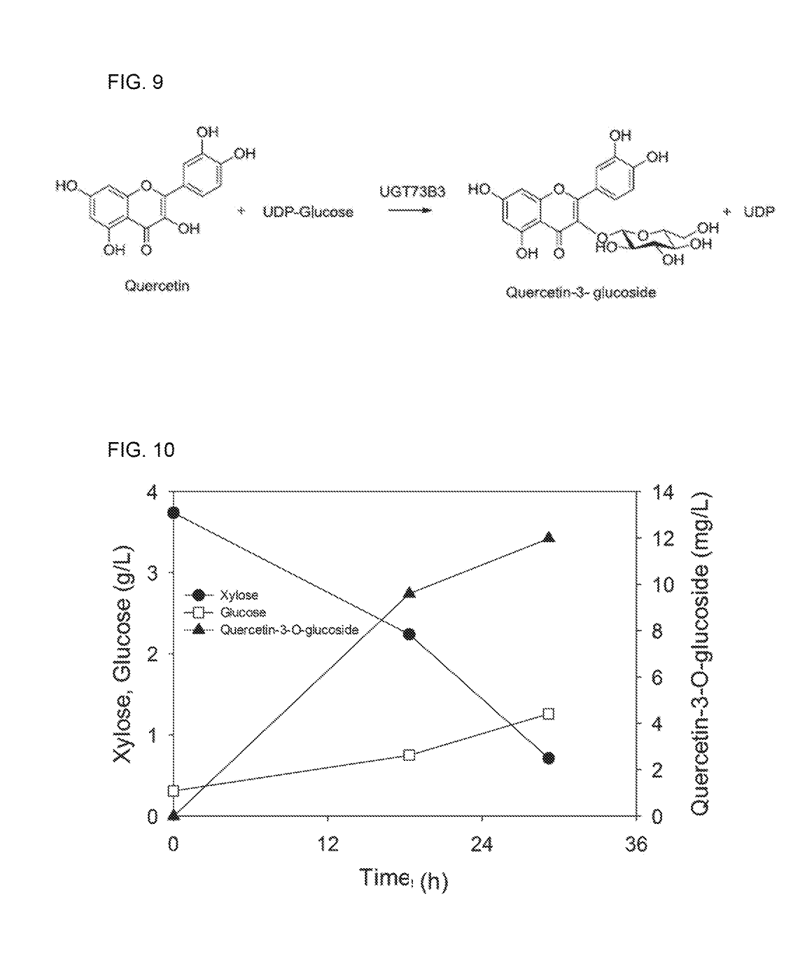

We next examined whether other metabolites of glucose-6P could be generated from a pentose. The flavonoid quercetin can be converted into the glucoside quercetin-3-O-glucoside by the glycotransferase enzyme UGT73B3 (Lim et al., 2004, Biotechnol. Bioeng.. 87: 623-631) as shown in FIG. 9, and the goal of this experiment was to derive this glucoside from xylose. UDP-glucose serves as a glucose donor to quercetin, and UDP-glucose is derived from glucose-6P through the enzymes phosphoglucomutase (mediating the conversion of glucose-6P to glucose-1P) and pyrophosphorylase (mediating the conversion of glucose-1P to UDP-glucose). No alteration in these genes was made, and we therefore relied on the native expression level of these genes under the growth conditions used.

The UGT73B3 gene from Arabidopsis thaliana was PCR cloned and digested with EcoRI and Kpnl, then subsequently cloned into vector pTrc99A to yield plasmid pTrc99A-UGT73B3. We transformed MEC143 with pTrc99A-UGT73B3 expressing this glycotransferase.

The medium used contained (per L): 13.3 g KH₂PO₄, 4.0 g (NH₄)₂HPO₄, 1.2 g MgSO₄·7H₂O, 13.0 mg Zn(CH₃COO)₂·2H₂O, 1.5 mg CuCl₂·2H₂O, 15.0 mg MnCl₂·4H₂O, 2.5 mg CoCl₂·6H₂O, 3.0 mg H₃BO₃, 2.5 mg Na₂MoO₄·2H₂O, 100 mg Fe(III)citrate, 8.4 mg Na₂EDTA·2H₂O, 1.7 g citric acid, 4.5 mg thiamine·HCl, and 4 g/L xylose. The medium also contained 100 mg/L ampicillin and 50 mg/L kanamycin.

MEC143/pTrc99A-UGT73B3 was first cultured in a 125 mL flask containing 10 mL medium. When the optical density (OD) reached 3, 2 mL was used to inoculate a 250 mL shake flask containing 50 mL of the same medium. These shake flasks were incubated at the studied temperature and 250 rpm (19 mm pitch). When the OD reached 1, 0.5 mM IPTG and quercetin dissolved in DMSO to provide a flask concentration of approximately 30 mg/L were added. The shake flasks were adjusted to an initial pH of 7.0 with 20% NaOH, and the culture was grown at 20° C.

To analyze for the glucoside, a 1 mL sample was centrifuged to obtain a supernatant and a cell pellet, which was resuspended in 0.2 mL DMSO to extract quercetin glucosides. The combined supernatant and extracted pellet fractions were centrifuged again, and the supernatant was analyzed by HPLC at 370 nm using a 5-μm C₁₈ column. Quercetin glucosides and quercetin were separated using a linear gradient of 20% to 80% acetonitrile in H₂O (with 0.1% tri-fluoroacetic acid) at 1 mL/min over 60 min.

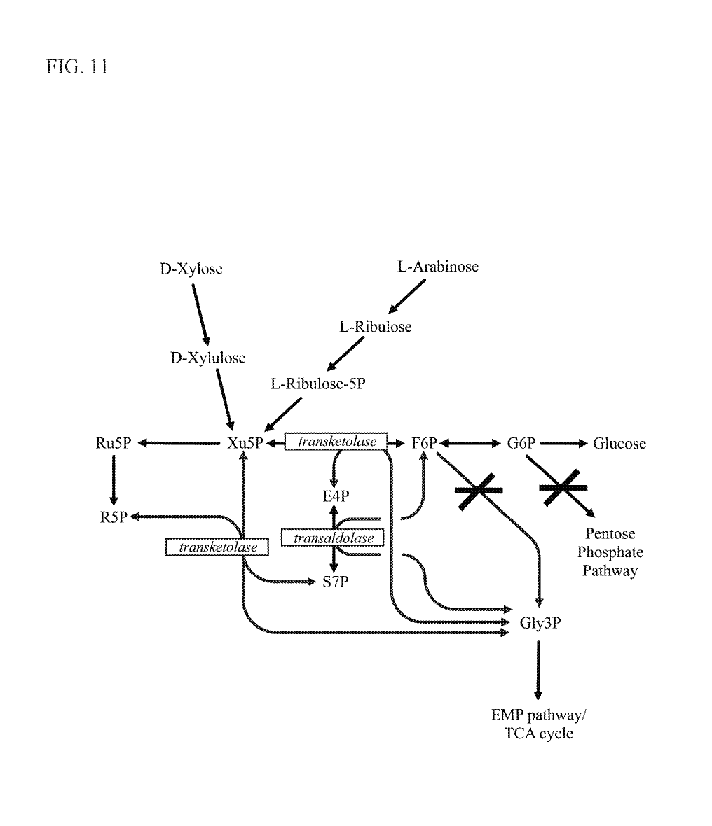

FIG. 10 shows the conversion of xylose into both glucose and quercetin-3-O-glucoside. Although significant glucose was formed from xylose (1.3 g/L glucose for a yield of 0.34 g/g), the yield of quercetin-3-O-glucoside on the basis of the small amount of quercetin supplied (30 mg/L) was 0.40 g/g. Despite the fact that no additional genetic optimization of the growth conditions or the pathway to the glucoside was made, this experiment demonstrates the ability of this process to convert pentoses into compounds metabolically derived from glucose-6P.

### Reference

Lim et al., 2004. Arabidopsis Glycosyltransferases as Biocatalysts in Fermentation for Regioselective Synthesis of Diverse Quercetin Glucosides. Biotechnology and Bioengineering. 87: 623-631.

### Example 6

### Influence of Phosphatases and Phosphate on the Formation of Glucose from Pentoses in Escherichia coli

Metabolically engineered Escherichia coli with deletions of the ptsG, manZ, glk, pfkA and zwf genes convert pentoses such as arabinose and xylose into glucose (see, e.g., Xia et al., 2012). The final step involves the dephosphorylation of glucose-6-phosphate.

The accumulation of glucose requires deletions in several genes involved both in the initial conversion of glucose (phosphotransferase enzymes capable of glucose uptake, ptsG and manZ genes, and glucokinase, glk), and in glucose-6-phosphate (glucose-6P) metabolism (6P-fructokinase I, pfkA, and glucose-6P 1-dehydrogenase, zwf). This transformation of five-carbon sugars to six-carbon glucose can occur because of the reversibility of transketolase and transaldolase, key enzymes of the pentose phosphate pathway (FIG. 11) which exchange monosaccharides of 3-7 carbon length and ultimately lead to fructose-6P and glyceraldehyde-3P:

xylose or arabinose+n ATP→2 fructose-6P+glyceraldehyde-3P+(n−3) ADP [9]

The quantity of ATP required for this conversion depends on the mechanism the cells use for pentose transport (Xia et al., 2015; Example I). The pfkA zwf gene deletions prevent the re-entry of the product fructose-6P (Eq. 9) into central metabolism by the Embden-Meyerhof-Parnas and pentose phosphate pathways. Since an additional knockout in either agp (glucose 1-phosphatase) orpgm (phosphoglucose mutase) failed to prevent glucose formation, while a deletion inpgi (phosphoglucose isomerase) eliminated glucose formation, the final step in the conversion of pentoses via fructose-6P to glucose is the dephosphorylation of glucose-6P:

glucose-6P→glucose+Pi [6]

Dephosphorylation is mediated by phosphatases, enzymes which typically act on multiple substrates. In E. coli, for example, glucose-6P has been shown to be dephosphorylated by alkaline phosphatase (Heppel et al., 1962) and by several haloacid dehalogenase-like phosphatases (Kuznetsova et al., 2006). Which enzyme is responsible for the final step (Eq. 6) in the conversion of pentoses to glucose is unknown.

Regardless of the enzyme responsible for the in vivo dephosphorylation of glucose-6P, Equation 6 also indicates that phosphate is a by-product of glucose production. This chemical equation therefore suggests, by simple mass action, that phosphate-limited conditions should favor the forward reaction forming glucose. Whereas batch culture of organisms provides cells with excess phosphate and other nutrients during the majority of growth, phosphate-limited conditions can readily be accomplished by growing cells under steady-state conditions in a medium in which phosphate is always exhausted. Similarly, during carbon-limited (and with excess phosphate) steady-state growth chemotrophic cells like E. coli would use the largest possible fraction of the carbon source for energy, resulting in the least amount of glucose accumulation.

The objectives of this study were to determine which enzymes mediate the dephosphorylation of glucose-6P to glucose during the formation of glucose from pentoses. Additionally, we sought to determine if a greater glucose yield could be obtained from xylose or arabinose by growing cells under phosphate-limited conditions. To this end, we examined six different phosphatases singly and in combination, and demonstrated that multiple phosphatases are responsible for the final conversion of glucose-6-phosphate to glucose. Overexpression of one phosphatase, HAD12 coded by the ybiV gene, resulted in 9-16% increase in glucose yield. Finally, growing cells under phosphate-limited conditions increased the glucose yield by 50% to 0.39 g glucose/g xylose, but did not improve glucose yield from arabinose (0.31 g/g). These observations can be explained by the different phosphate demands resulting from E. coli metabolizing xylose compared to arabinose.

### Materials and Methods

### Bacterial Strains and Construction

Numerous strains of Escherichia coli were constructed from MEC149 (MG1655 ptsG manZ glk pfkA zwf) as listed in Table 2. These strains were constructed by transduction of MEC149 and the corresponding Keio (FRT)Kan deletions strains using P1 bacteriophage virus (Baba et al., 2006), and if necessary, curing the Kan(R) using the pCP20 plasmid, which contains a temperature-inducible FLP recombinase as well as a temperature-sensitive replicon (Datsenko and Wanner, 2000). Polymerase chain reactions were conducted to confirm each strain.

TABLE 2
E. coli strains used in this study.

| Strain | Genotype | Reference |
|---|---|---|
| MEC143 | MG1655 ΔptsG763::(FRT) ΔmanZ743::(FRT) | Xia et al., 2015; |
| | Δglk-726::(FRT) ΔpfkA775::(FRT) Δzwf-777::Kan | Example 1 |
| MEC149 | MG1655 ΔptsG763::(FRT) ΔmanZ743::(FRT) | This study |
| | Δglk-726::(FRT) ΔpfkA775::(FRT) Δzwf-777::(FRT) | |
| MEC149 ybiV | MEC149 ΔybiV722::Kan | This study |
| MEC149 yigL | MEC149 ΔyigL771::Kan | This study |
| MEC149 yfbT | MEC149 ΔyfbT775::Kan | This study |
| MEC149 yniC | MEC149 ΔyniC726::Kan | This study |
| MEC149 yidA | MEC149 ΔyidA733::Kan | This study |
| MEC149 phoA | MEC149 ΔphoA748::Kan | This study |
| MEC149 ybiV yidA | MEC149 ΔybiV722::(FRT) ΔyidA733::Kan | This study |
| MEC149 ybiV yigL | MEC149 ΔybiV722::(FRT) ΔyigL771::Kan | This study |
| MEC149 ybiV yigL yidA | MEC149 ΔybiV722::(FRT) ΔyigL771::(FRT) | This study |
| | ΔyidA733::Kan | |

The gene ybiV coding the haloacid dehalogenase-like phosphatase 12 (HAD12) in E. coli (Roberts et al., 2005; Kuznetsova et al., 2006) was PCR amplified by the gene specific primers 5′-GGGAAAGGTACCATGAGCGTAAAAGTTATCGTCACAG-3′ (SEQ ID NO:3) (forward) and 5′- GGGAAATCTAGATCAGCTGTTAAAAGGGGATGTG-3′ (SEQ

ID NO:4) (reverse) using genomic DNA of wild-type MG1655 as template. The PCR product (794 bp) was purified and digested with kpnl and xbal and ligated into the expression vector pZE12 which was also digested with same endonuclease enzymes. This plasmid-gene cassette pZE12-ybiVwas transformed into MEC143 resulting in E. coli MEC143/pZE12-ybiV.

### Growth Medium and Culture Conditions

The defined medium used for the shake flask experiments contained (per liter): 1.70 g citric acid, 13.30 g KH₂PO₄, 4.00 g (NH₄)₂HPO₄, 1.2 g MgSO₄·7H₂O, 13 mg Zn(CH₃COO)₂·2H₂O, 1.5 mg CuCl₂·2H₂O, 15 mg MnCl₂·4H₂O, 2.5 mg CoCl₂·6H₂O, 3.0 mg H₃BO₃, 2.5 mg Na₂MoO₄·2H₂O, 100 mg Fe(III) citrate, 4.5 mg thiamine·HCl, 8.4 mg Na₂(EDTA)·2H₂O, and 5.0 g D-xylose or L-arabinose. The pH was adjusted to 7.0 with 30% (w/v) NaOH. Cells were routinely stored on Lysogeny Broth (LB) agar plates inoculated to 3 mL LB in 15 mL tube from which 1 mL was transferred to 20 mL defined medium in a 125 mL shake flask. From this flask 1 mL was transferred to the 50 mL defined medium in a 250 mL shake flask used for these studies. The flasks were incubated at 37° C. with an agitation of 250 rpm and for the further analysis samples were stored at −20° C. Shake flask studies were replicated 3 or more times, and statistical analyses were completed using Student's t-test (two-tailed, equal variance), and p<0.10 was considered the criterion for significance.

Continuous processes of 1 L volume were conducted as phosphate-limited or carbon limited chemostats in a 2.5 L fermenter (Bioflo 2000, New Brunswick Scientific Co. Edison, N.J., USA) using MEC143 strain with xylose or arabinose. Carbon-limited chemostats used the medium described above. In the medium for phosphate-limited chemostats, 3.25 g/L NH₄Cl (60.8 mM N) replaced for (NH₄)₂HPO₄, and 0.12 g/L KH₂PO₄ (0.74 mM P) was used as the sole phosphate source. These processes were conducted at dilution rate of 0.15 h⁻¹ at 37° C. with an air flowrate of 1.0 L/min, an agitation of 500 rpm and a pH of 7.0. When appropriate for the strain, 40 mg/L (LB) or 100 mg/L (defined medium) kanamycin and 100 mg/L (LB) or 50 mg/L (defined) ampicillin were used.

### Analytical Methods

The cell growth was monitored using optical density at 600 nm (OD) (UV-650 spectrophotometer, Beckman Instruments, San Jose, Calif.). Liquid chromatography with a refractive index detector and a Coregel 64-H ion-exclusion column (Transgenomic Ltd., Glasgow, United Kingdom) using a mobile phase of 4 mN H₂SO₄ was used for analysis of sugars as described previously (Eiteman and Chastain, 1997). For dry cell weight measurement, three 10.0 mL samples were centrifuged (8400×g, 10 min), the pellets washed by vortex mixing with 30 mL deionized water three times with centrifugation, and then the pellets dried at 60° C. for 24 h. Phosphorus concentration was analyzed using the ascorbic acid reduction method (Murphy and Riley, 1977; Eaton et al., 2005).

### Results and Discussion

Enzymes which dephosphorylate glucose-6-phosphate

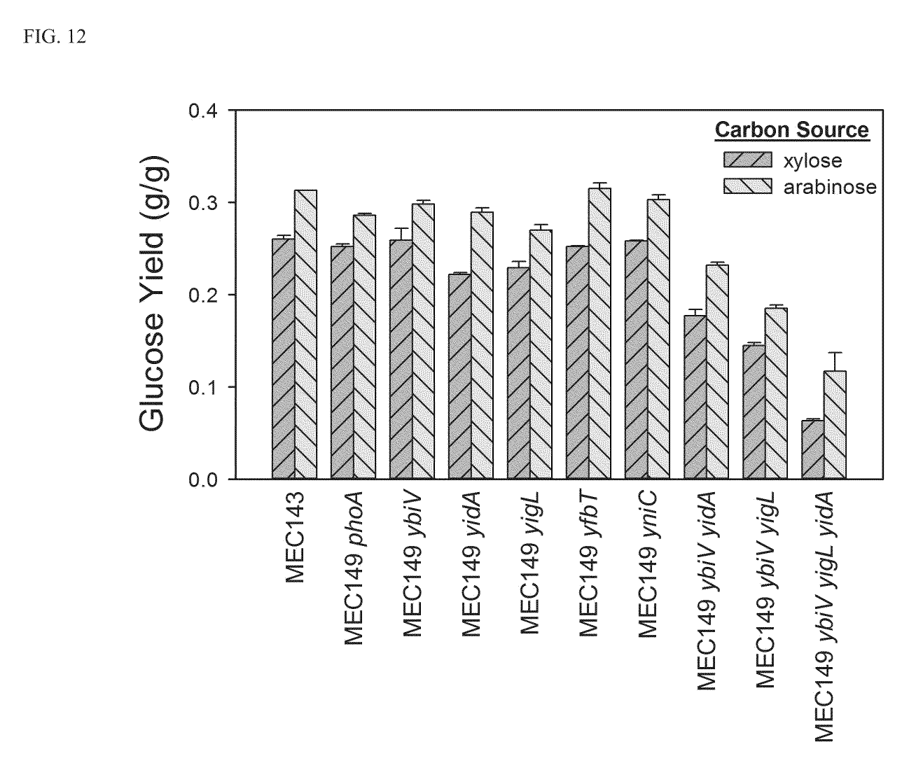

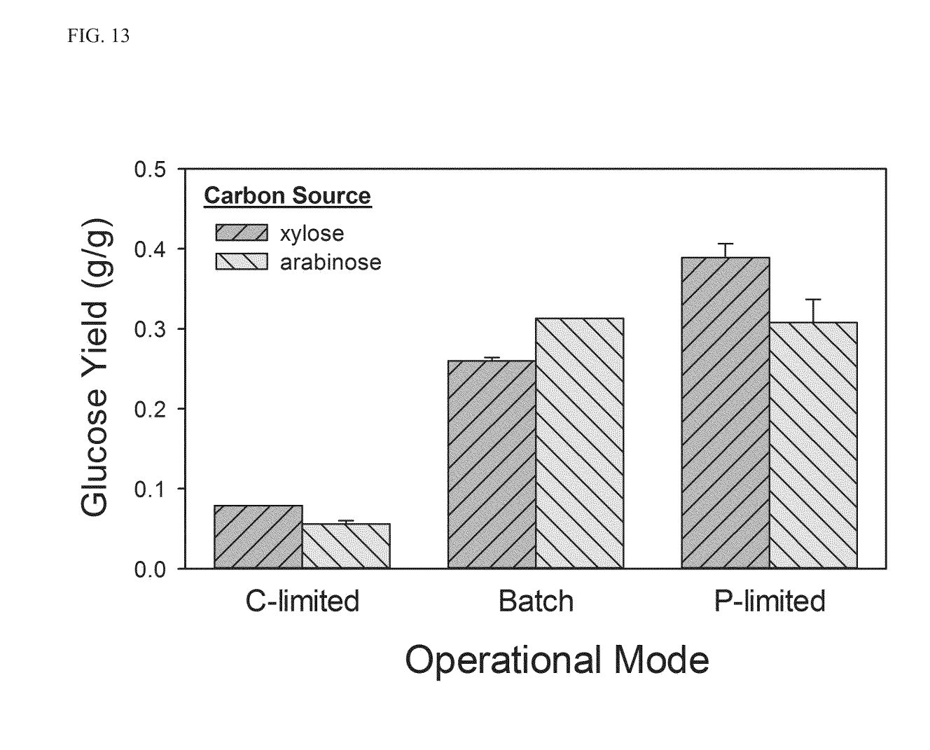

Escherichia coli MEC143 has deletions in the genes which allow glucose uptake (ptsG, manZ, glk), and the genes which are involved in the metabolism of glucose-6P and fructose-6P (zwf, pfkA), and therefore MEC143 accumulates glucose when grown on either xylose or L-arabinose (Xia et al., 2015; Example 1). Mass glucose yields of 0.260 g glucose/g xylose and 0.313 g glucose/g L-arabinose were observed in shake flask studies using 5 g/L of either pentose (Table 2). A previous study established that glucose formation occurs by the dephosphorylation of glucose-6P (Xia et al., 2015; Example 1). Since several phosphatases which act on glucose-6P have been identified in E. coli including alkaline phosphatase (Heppel et al., 1962) and various HAD phosphatases (Kuznetsova et al., 2006), we hypothesized that one or more of these enzymes was responsible for glucose formation in MEC143. In order to establish the phosphatases responsible for the conversion of glucose-6P to glucose in MEC 143, we first examined whether a deletion in any one of these genes alone was would affect glucose formation. Deletions in either the phoA, ybiV, yfbT or yniC genes did not significantly reduce the glucose yield from xylose and arabinose (FIG. 12). A deletion in either yidA or yigL reduced the yields slightly to approximately 0.22 g glucose/g xylose and 0.27-0.29 g glucose/g L-arabinose. Nevertheless, a deletion in any one gene was insufficient to prevent glucose formation from either xylose or arabinose. Thus, these results suggest that no one phosphatase is exclusively responsible for the conversion of glucose-6P to glucose in MEC143.

Because a single deletion in any of the genes coding alkaline phosphatase or the HAD phosphatases was insufficient to prevent glucose accumulation, we next examined whether knocking out multiple phosphatases would further reduce glucose formation. In particular we examined combinations of knockouts of those genes which showed a small but significant reduction in glucose yield when knocked out singly (FIG. 12). For example, the double knockout ybiV yidA resulted in a yield of 0.177 g glucose/g xylose and 0.232 g glucose/g L-arabinose, while the triple knockout ybiV yidA yigL resulted in a yield of 0.063 g glucose/g xylose and 0.117 g glucose/g L-arabinose (FIG. 12). These multiple additional knockouts increased the lag phase and reduced the growth rate E. coli on both xylose and L-arabinose, generally by a factor of about two. Our conclusion is these phosphatases (YbiV, YidA and YigL) and others are indeed responsible for the conversion of glucose-6P to glucose which leads to glucose formation in E. coli MEC143. However, the results also support the conclusion that no one phosphatase is even primarily responsible for the in vivo dephosphorylation of glucose-6P. Furthermore, since the phosphatases are known to act on many other organic phosphates (Heppel et al., 1962; Kuznetsova et al., 2006), they likely mediate other important conversions, which makes them collectively essential for cell health.

Since one or more HAD phosphatases are involved in dephosphorylation of glucose-6P to glucose, we also examined whether overexpression of a phosphatase would conversely increase the glucose formation from pentoses. We selected HAD12 coded by ybiV because this enzyme has high observed values for both the k_{cat} (22 s⁻¹) and the pseudo-first order rate constant (k_{cat}/K_{M}) of 6900 M⁻¹s⁻¹ (Kuznetsova et al., 2006). In shake flask studies using MEC143/pZE12-ybiV we obtained yields of 0.288 g glucose/g xylose and 0.342 g glucose/g L-arabinose, 16% and 9% greater than the yields observed with MEC143. This increase in yield is particularly significant considering the increased ATP requirement which would occur in this strain overexpressing a protein from a plasmid. Our conclusion is that one method to increase the yield of a hexose from pentoses is to enhance the final dephosphorylation (Eq. 6).

### Effect of Phosphate- or Carbon-Limitation

The shake flask studies operated as batch reactors, wherein all nutrients required for cell growth were supplied in excess at the onset of cell growth. The shake flask results (i.e., FIG. 12) therefore represent cellular responses under maximal growth conditions under conditions of excess phosphorus and carbon. The final step in the overall conversion of pentoses to glucose is the dephosphorylation of glucose-6P accompanied by the formation of inorganic phosphate (Eq. 2). Thus, by mass action a reduction in the availability of phosphate should promote the forward direction toward glucose. Although a batch operation does not allow for nutrient limitation, a chemostat operated at steady-state allows cell growth at a defined growth rate under a predetermined nutrient limitation. We hypothesized that phosphate limitation should increase glucose yield from pentose by MEC143. In contrast, under carbon-limited conditions, the glucose yield will be reduced compared to carbon-excess conditions of a batch culture, since cells must maximally metabolize that carbon for energy and biomass formation. To examine glucose formation under these two contrasting conditions, we completed several continuous, steady-state experiments under both phosphate limitation and carbon limitation (FIG. 13). As expected, carbon limitation significantly reduced the glucose yield from either pentose to 0.05-0.08 g/g, while phosphate limitation increased the glucose yield to 0.39 g glucose/g xylose, a 50% increase in glucose formation. Interestingly, we observed no significant change in glucose yield from arabinose (FIG. 13). Under phosphate-limited, we determined the yield coefficient (Y_{X/P}) of E. coli cells to be 58.3 g DCW/g P. Considering that both pentoses must use the final dephosphorylation of glucose-6P to generate glucose (Eq. 6), no specific explanation is provided for the large increase in glucose yield from xylose compared to arabinose. We also note though that in all shake flask studies the yield of glucose from arabinose was greater than the yield of glucose from xylose (FIG. 12). Thus, we speculate that a difference exists in the cellular “phosphate budget” between xylose and arabinose. This possibility is supported by the different routes by which the two pentoses are transported and enter the PP pathway in E. coli. For xylose, an ATP-binding dependent system (Ahlem et al., 1982) appears to predominate under normal growth conditions (Hasona et al. 2004), indicating that D-xylose uptake generally demands energy directly in the form of ATP. In contrast for arabinose, a low affinity, ATP-independent system appears to predominate (Daruwalla et al., 1981). Thus, cells may respond differently to phosphate limitation when the demand for ATP is low for the initial uptake and initial phosphorylation of the carbon/energy source. Our results nevertheless demonstrate that in certain cases a phosphate-limited process is able to increase the yield of glucose and potentially other products derived from glucose-6P. This same strategy could potentially be employed to increase the yield of other products whenever the final biochemical step involves dephosphorylation.

In summary, the final enzymatic step involved in the conversion of xylose or L-arabinose to glucose in E. coli unable to metabolize glucose is the dephosphorylation of glucose-6P to glucose (Eq. 2). Several general phosphatases share in mediating this conversion, as well as other critical cellular conversions, and the yield of glucose can be affected by the deletion or overexpression of these general phosphatases. Additionally, by simple mass action the formation of glucose is increased by growing cells in a phosphate-limited environment. Thus, our results shed light on conversions between monosaccharides in the upper metabolism of E. coli and also provide guidance on operational conditions to influence these conversions.

### References

Ahlem, C., W. Huisman, G. Heslund, and A. S. Dahms. 1982. Purification and properties of a periplasmic D-xylose-binding protein from Escherichia coli K-12. J Biol. Chem. 257:2926-2931.

Baba, T., T. Ara, M. Hasegawa, Y. Takaki, Y. Okumura, M. Baba, K. A. Datsenko, M. Tomita, B. L. Wanner, H. Mori. 2006. Construction of Escherichia coli K-12 in-frame, single-gene knockout mutants: the Keio collection. Mol. Syst. Biol. 1-11.

Daruwalla, K. R., A. T. Paxton, and P. J. F. Henderson. 1981. Energization of the transport systems for arabinose and comparison with galactose transport in Escherichia coli, Biochem. 1 200:611-627.

Datsenko, K. A., B. L. Wanner. 2000. One-step inactivation of chromosomal genes in Escherichia coli K-12 using PCR products. Proc. Natl. Acad. Sci. USA 97:6640-6645.

Eaton, A. D., L. S. Clesceri, E. W. Rice, A. E. Greenberg, M. H. Franson, eds. 2005. Standard Methods for the Examination of Water and Wastewater: 21^{st} ed. Amer. Public

Health Assoc. Washington, DC; Water Environment Federation, Alexandria, Va.; and Amer. Water Works Assoc., Denver, Colo.

Eiteman, M. A., M. J. Chastain. 1997. Optimization of the ion-exchange analysis of organic acids from fermentation. Anal. Chim. Acta 338:69-75.

Hasona, A., T. Kim, F. G. Healy, L. O. Ingram, and K. T. Shanmugam. 2004. Pyruvate formate lyase and acetate kinase are essential for anaerobic growth of Escherichia coli on xylose. J. Bacteriol. 186:7593-7600.

Heppel, L. A., D. R. Harkness, R. J. Hilmoe. 1962. A study of the substrate specificity and other properties of the alkaline phosphatase of Escherichia coli. J. Biol. Chem. 237(3):841-846.

Kuznetsova E., M. Proudfoot, C. F. Gonzalez, G. Brown, M. V. Omelchenko, Y. I. Wolf, H. Mori, H., A. V. Savchenko, C. H. Arrowsmith, E. V. Koonin, A. M. Edwards, A. F. Yakunin. 2006. Genome-wide analysis of substrate specificities of the Escherichia coli haloacid dehalogenase-like phosphatase family. J. Biol. Chem. 281:36149-3616.

Murphy, J., J. R. Riley. 1977. A modfied single solution method for the dtermination of phosphate in natural waters. Anal. Chem. 27:31-36.

Roberts, A., S. Y. Lee, E. McCullagh, R. E. Silversmith, D. E. Wemmer. 2005. YbiV from Escherichia coli K12 is a HAD phosphatase. Proteins: Structure, Function, and Bioinformatics, 58(4):790-801.

Xia, T., Q. Han, W. V. Costanzo, Y. Zhu, J. L. Urbauer, M. A. Eiteman. 2015. Accumulation of D-glucose from pentoses by metabolically engineering Escherichia coli. Appl. Environ. Microbiol. 81:3387-3394.

The complete disclosure of all patents, patent applications, and publications, and electronically available material (including, for instance, nucleotide sequence submissions in, e.g., GenBank and IUBMB, and amino acid sequence submissions in, e.g., SwissProt, PIR, PRF, PDB, and translations from annotated coding regions in GenBank and IUBMB) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A metabolically engineered cell which accumulates a compound comprising a metabolite of glucose-6P or fructose-6P, the cell comprising:
(a) deletion or inactivation of at least one gene involved in uptake of a hexose so as to disrupt or prevent metabolism the hexose; and
(b) deletion or inactivation of at least one gene involved in the metabolism of fructose-6P so as to divert a carbon source to the glucose-6P or fructose-6P metabolite;
wherein the cell accumulates the compound comprising the glucose-6P or fructose-6P metabolite.

2. The metabolically engineered cell of claim 1, further comprising:
(c) deletion or inactivation of at least one gene involved in the pentose phosphate pathway so as to divert a carbon source to the glucose-6P or fructose-6P metabolite.

3. The metabolically engineered cell of claim 1, wherein the cell overexpresses a phosphatase.

4. The metabolically engineered cell of claim 1 wherein the accumulated compound comprises the hexose.

5. The metabolically engineered cell of claim 1, wherein (a) comprises deletion or inactivation of at least one gene involved in uptake of the accumulated compound.

6. The metabolically engineered cell of claim 1, wherein the hexose comprises glucose, fructose, or mannose.

7. The metabolically engineered cell of claim 1, wherein the accumulated compound comprises glucose or mannose.

8. The metabolically engineered cell of claim 1, wherein the accumulated compound comprises a glucoside.

9. The metabolically engineered cell of claim 1, wherein the accumulated compound comprises a glucuronic acid.

10. The metabolically engineered cell of claim 1, wherein the accumulated compound comprises hyaluronic acid.

11. The metabolically engineered cell of claim 1, comprising deletion or inactivation of ptsG, at least one of manX, manY or manZ, and glk or their counterparts.

12. The metabolically engineered cell of claim 1, further comprising deletion or inactivation of one or both of pfkA and zwf.

13. The metabolically engineered cell of claim 1, wherein the carbon source is a pentose or a sugar alcohol.

14. The metabolically engineered cell of claim 13, wherein the pentose is xylose or arabinose.

15. The metabolically engineered cell of claim 13, wherein the sugar alcohol is glycerol.

16. The metabolically engineered cell of claim 1, which is a bacterial cell.

17. The metabolically engineered cell of claim 16, wherein the bacterial cell is an Escherichia coli cell.

18. A method for producing a compound comprising a metabolite of glucose-6P or fructose-6P, the method comprising culturing the cell of claim 1 in the presence of a carbon source under conditions to allow the cell to accumulate the compound.

19. The method of claim 18, wherein the accumulated compound comprises a hexose comprising glucose, mannose, or fructose.

20. The method of claim 18, wherein the carbon source is a pentose or a sugar alcohol.

21. The method of claim 20, wherein the pentose is xylose or arabinose.

22. The method of claim 20, wherein the sugar alcohol is glycerol.

23. The method of claim 18, wherein the cell is cultured in a phosphate-limited medium.

24. The method of claim 18 wherein the cell is a bacterial cell.

25. The method of claim 24, wherein the bacterial cell is an E. coli cell.

26. The method of claim 18, further comprising isolating the compound from the cell or from the cell culture medium.

27. The method of claim 26, further comprising purifying the compound.


SEQUENCE LISTING (4 sequences)

SEQ ID NO: 1 - DNA, 38 bp, artificial
  Primer
gggaaagaattcaaaaacatcaatccaacgcagaccgc

SEQ ID NO: 2 - DNA, 36 bp, Artificial
  Primer
gggaaaggatccttaaccgcgccacgctttatagcg

SEQ ID NO: 3 - DNA, 37 bp, Artificial
  Primer
gggaaaggtaccatgagcgtaaaagttatcgtcacag

SEQ ID NO: 4 - DNA, 34 bp, Artificial
  Primer
gggaaatctagatcagctgttaaaaggggatgtg